(19) United States
(12) Patent Application Publication
(10) Pub. No.: US 2005/0288346 A1
(43) Pub. Date: Dec. 29, 2005

(54) Acetophenone potentiators of metabotropic glutamate receptors

(76) Inventors: Rowena V. Cube, San Diego, CA (US); Anthony B. Pinkerton, San Diego, CA (US); Jean-Michel Vernier, San Diego, CA (US); Xiumin Zhao, San Diego, CA (US)

Correspondence Address: MERCK AND CO., INC, P O BOX 2000, RAHWAY, NJ, 07065-0907, US

(21) Appl. No.: 10/524,704

(22) PCT Filed: Aug. 22, 2003
(86) PCT No.: PCT/US03/26377

§ 371 Date: Feb. 14, 2005

Related U.S. Application Data
(60) Provisional application No. 60/405,908, filed on Aug. 26, 2002.

(51) Int. Cl. A61K 31/41; C07D 257/02

(52) U.S. Cl. 514/381; 548/253

(57) ABSTRACT

The present invention is directed to compounds which are potentiators of metabotropic glutamate receptors, including the mGluR2 receptor, and which are useful in the treatment or prevention of neurological and psychiatric disorders associated with glutamate dysfunction and diseases in which metabotropic glutamate receptors are involved. The invention is also directed to pharmaceutical compositions comprising these compounds and the use of these compounds and compositions in the prevention or treatment of such diseases in which metabotropic glutamate receptors are involved.

### BACKGROUND OF THE INVENTION

The excitatory amino acid L-glutamate (sometimes referred to herein simply as glutamate) through its many receptors mediates most of the excitatory neurotransmission within the mammalian central nervous system (CNS). The excitatory amino acids, including glutamate, are of great physiological importance, playing a role in a variety of physiological processes, such as long-term potentiation (learning and memory), the development of synaptic plasticity, motor control, respiration, cardiovascular regulation, and sensory perception.

Glutamate acts via at least two distinct classes of receptors. One class is composed of the ionotropic glutamate (iGlu) receptors that act as ligand-gated ionic channels. Via activation of the iGlu receptors, glutamate is thought to regulate fast neuronal transmission within the synapse of two connecting neurons in the CNS. The second general type of receptor is the G-protein or second messenger-linked “metabotropic” glutamate (mGluR) receptor. Both types of receptors appear not only to mediate normal synaptic transmission along excitatory pathways, but also participate in the modification of synaptic connections during development and throughout life. Schoepp, Bockaert, and Sladeczek, Trends in Pharmacol. Sci., 11, 508 (1990); McDonald and Johnson, Brain Research Reviews, 15, 41 (1990).

The present invention relates to potentiators of mGlu receptors, in particular mGluR2 receptors. The mGluR receptors belong to the Type m G-protein coupled receptor (GPCR) superfamily. This superfamily of GPCR′sf including the calcium-sensing receptors, GABAB receptors and pheromone receptors, which are unique in that they are activated by binding of effectors to the amino-terminus portion of the receptor protein. The mGlu receptors are thought to mediate glutamate's demonstrated ability to modulate intracellular signal transduction pathways. Ozawa, Kamiya and Tsuzuski, Prog. Neurobio., 54, 581 (1998). They have been demonstrated to be localized both pre- and post-synaptically where they can regulate neurotransmitter release, either glutamate or other neurotransmitters, or modify the post-synaptic response of neurotransmitters, respectively.

At present, there are eight distinct mGlu receptors that have been positively identified, cloned, and their sequences reported. These are further subdivided based on their amino acid sequence homology, their ability to effect certain signal transduction mechanisms, and their known pharmacological properties. Ozawa, Kamiya and Tsuzuski, Prog. Neurobio., 54, 581 (1998). For instance, the Group I mGluR receptors, which include the mGlu1R and mGlu5R, are known to activate phospholipase C (PLC) via Gaq-proteins thereby resulting in the increased hydrolysis of phosphoinositides and intracellular calcium mobilization. There are several compounds that are reported to activate the Group I mGlu receptors including DHPG, (R/S)-3,5-dihydroxyphenylglycine. Schoepp, Goldworthy, Johnson, Salhoff and Baker, J. Neurochem., 63, 769 (1994); Ito, et al., keurorep., 3, 1013 (1992). The Group II mGlu receptors consist of the two distinct receptors, mGluR2 and mGluR3 receptors. Both have been found to be negatively coupled to adenylate cyclase via activation of Gai-protein. These receptors can be activated by a selective compound such as 1S,2S,SR,6S-2 aminobicyclo[3.1.0]hexane-2,6-dicarboxylate. Monn, et al., J. Med. Chem., 40, 528 (1997); Schoepp, et al., Neuropharmacol., 36, 1 (1997). Similarly, the Group m mGlu receptors, including mGluR4, mGluR6, mGluR7 and mGluR8, are negatively coupled to adenylate cyclase via Gai and are potently activated by L-AP4 (L-(+)-2-amino-4-phosphonobutyric acid). Schoepp, Neurochem. Int., 24,439 (1994).

It has become increasingly clear that there is a link between modulation of excitatory amino acid receptors, including the glutamatergic system, through changes in glutamate release or alteration in postsynaptic receptor activation, and a variety of neurological and psychiatric disorders. e.g. Monaghan, Bridges and Cotman, Ann. Rev. Pharmacol. Toxicol., 29, 365-402 (1989); Schoepp and Sacann, Neurobio. Aging, 15, 261-263 (1994); Meldrum and Garthwaite, Tr. Pharmacol. Sci., 11, 379-387 (1990). The medical consequences of such glutamate dysfunction makes the abatement of these neurological processes an important therapeutic goal.

### SUMMARY OF THE INVENTION

The present invention is directed to compounds which are potentiators of metabotropic glutamate receptors, including the mGluR2 receptor, and which are useful in the treatment or prevention of neurological and psychiatric disorders associated with glutamate dysfunction and diseases in which metabotropic glutamate receptors are involved. The invention is also directed to pharmaceutical compositions comprising these compounds and the use of these compounds and compositions in the prevention or treatment of such diseases in which metabotropic glutamate receptors are involved.

### DETAILED DESCRIPTION OF THE INVENTION

The present invention is directed to compounds of the formula I: 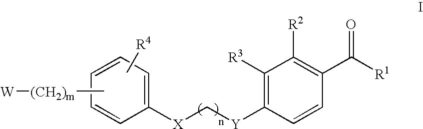
wherein:
W is selected from the group consisting of:
   (1) tetrazolyl,
   (2) CO₂H,
   (3) NHSO₂C₁₋₆alkyl, and
   (4) CONHCO—C₁₋₆alkyl;
X is selected from the group consisting of:
   (1) —O—,
   (2) —S—, and
   (3) —NH—,
   (4) —N(C₁₋₆alkyl)—,
   (5) a bond;
Y is selected from the group consisting of:
   (1) —O—, and
   (2) —S—;
R¹ is selected from the group consisting of:
   (1) C₁₋₆alkyl, which is unsubstituted or substituted with a substituent selected from:
      (a) halogen,
      (b) hydroxyl, and
      (c) phenyl, wherein the phenyl is unsubstituted or substituted with 1-5 substituents independently selected from halogen, cyano, CF3, hydroxyl, C₁₋₆alkyl, and OC₁₋₆alkyl,
   (2) C₃₋₇cycloalkyl, which is unsubstituted or substituted with halogen, hydroxyl or phenyl, and
   (3) phenyl, wherein the phenyl is unsubstituted or substituted with 1-5 substituents independently selected from halogen, hydroxyl, cyano, CF3, C₁₋₆alkyl, and OC₁₋₆alkyl, wherein the C₁₋₆alkyl and OC₁₋₆alkyl are linear or branched and optionally substituted with 1-5 halogen;
R² is selected from the group consisting of:
   (1) hydroxyl,
   (2) halogen,
   (3) OC₁₋₆alkyl, and
   (4) C₁₋₆alkyl, which is unsubstituted or substituted with halogen, hydroxyl or phenyl;
R³ is selected from the group consisting of:
   (1) C₁₋₆alkyl, which is unsubstituted or substituted with halogen, hydroxyl or phenyl, and
   (2) halogen, and
   (3) hydrogen;
R⁴ is selected from the group consisting of:
   (1) hydrogen,
   (2) halogen, and
   (3) C₁₋₆alkyl;
m is an integer selected from 0, 1, 2 and 3;
n is an integer selected from 0, 1, 2, 3, 4, 5 and 6;
   and pharmaceutically acceptable salts thereof and individual diastereomers thereof.

A first embodiment of the present invention includes compounds wherein W is tetrazolyl.

A second embodiment of the present invention includes compounds wherein W is CO₂H.

An embodiment of the present invention includes compounds wherein X is —O—.

An embodiment of the present invention includes compounds wherein Y is —O—.

An embodiment of the present invention includes compounds wherein X is a bond and Y is —O—.

An embodiment of the present invention includes compounds wherein R¹ is C₁₋₆alkyl.

An embodiment of the present invention includes compounds wherein R¹ is C₅₋₆cycloalkyl.

An embodiment of the present invention includes compounds wherein R¹ is CH₃.

An embodiment of the present invention includes compounds wherein R¹ is CH(CH₃)₂.

An embodiment of the present invention includes compounds wherein R¹ is CH₂CH₃.

An embodiment of the present invention includes compounds wherein R¹ is CH₂CH₂CH₃.

An embodiment of the present invention includes compounds wherein R¹ is cyclopentyl.

An embodiment of the present invention includes compounds wherein R¹ is CH₂-cyclopentyl.

An embodiment of the present invention includes compounds wherein R¹ is phenyl.

An embodiment of the present invention includes compounds wherein R¹ is CH_{2phenyl}.

An embodiment of the present invention includes compounds wherein R² is hydroxyl.

An embodiment of the present invention includes compounds wherein R² is chloro.

An embodiment of the present invention includes compounds wherein R³ is C₁₋₆alkyl.

An embodiment of the present invention includes compounds wherein R³ is CH₃.

An embodiment of the present invention includes compounds wherein R³ is CH₂CH₃.

An embodiment of the present invention includes compounds wherein R³ is CH₂CH₂CH₃.

An embodiment of the present invention includes compounds wherein R³ is chloro.

An embodiment of the present invention includes compounds wherein R⁴ is hydrogen or bromo.

An embodiment of the present invention includes compounds wherein m is 0.

An embodiment of the present invention includes compounds wherein m is 1.

An embodiment of the present invention includes compounds wherein n is 1.

An embodiment of the present invention includes compounds wherein n is 2.

An embodiment of the present invention includes compounds wherein n is 3.

An embodiment of the present invention includes compounds wherein n is 4.

The compounds of the present invention are potentiators of metabotropic glutamate (mGluR) receptor function, in particular they are potentiators of mGluR2 receptors. That is, the compounds of the present invention do not appear to bind at the glutamate recognition site on the mGluR receptor, but in the presence of glutamate or a glutamate agonist, the compounds of the present invention increase mGluR receptor response. The present potentiators are expected to have their effect at mGluR receptors by virtue of their ability to increase the response of such receptors to glutamate or glutamate agonists, enhancing the function of the receptors. It is recognized that the compounds of the present invention would be expected to increase the effectiveness of glutamate and glutamate agonists of the mGluR2 receptor. Thus, the potentiators of the present invention are expected to be useful in the treatment of various neurological and psychiatric disorders associated with glutamate dysfunction described to be treated herein and others that can be treated by such potentiators as are appreciated by those skilled in the art.

The compounds of the present invention may contain one or more asymmetric centers and can thus occur as racemates and racemic mixtures, single enantiomers, diastereomeric mixtures and individual diastereomers. Additional asymmetric centers may be present depending upon the nature of the various substituents on the molecule. Each such asymmetric center will independently produce two optical isomers and it is intended that all of the possible optical isomers and diastereomers in mixtures and as pure or partially purified compounds are included within the ambit of this invention. The present invention is meant to comprehend all such isomeric forms of these compounds. Formula I shows the structure of the class of compounds without preferred stereochemistry.

The independent syntheses of these diastereomers or their chromatographic separations may be achieved as known in the art by appropriate modification of the methodology disclosed herein. Their absolute stereochemistry may be determined by the x-ray crystallography of crystalline products or crystalline intermediates which are derivatized, if necessary, with a reagent containing an asymmetric center of known absolute configuration.

If desired, racemic mixtures of the compounds may be separated so that the individual enantiomers are isolated. The separation can be carried out by methods well known in the art, such as the coupling of a racemic mixture of compounds to an enantiomerically pure compound to form a diastereomeric mixture, followed by separation of the individual diastereomers by standard methods, such as fractional crystallization or chromatography. The coupling reaction is often the formation of salts using an enantiomerically pure acid or base. The diasteromeric derivatives may then be converted to the pure enantiomers by cleavage of the added chiral residue. The racemic mixture of the compounds can also be separated directly by chromatographic methods utilizing chiral stationary phases, which methods are well known in the art.

Alternatively, any enantiomer of a compound may be obtained by stereoselective synthesis using optically pure starting materials or reagents of known configuration by methods well known in the art.

As appreciated by those of skill in the art, halo or halogen as used herein are intended to include fluoro, chloro, bromo and iodo. Similarly, C₁₋₆, as in C₁₋₆alkyl is defined to identify the group as having 1, 2, 3, 4, 5 or 6 carbons in a linear or branched arrangement, such that C₁₋₈alkyl specifically includes methyl, ethyl, n-propyl, iso-propyl, n-butyl, iso-butyl, tert-butyl, pentyl, and hexyl. A group which is designated as being independently substituted with substituents may be independently substituted with multiple numbers of such substituents.

The term “pharmaceutically acceptable salts” refers to salts prepared from pharmaceutically acceptable non-toxic bases or acids including inorganic or organic bases and inorganic or organic acids. Salts derived from inorganic bases include aluminum, ammonium, calcium, copper, ferric, ferrous, lithium, magnesium, manganic salts, manganous, potassium, sodium, zinc, and the like. Particularly preferred are the ammonium, calcium, magnesium, potassium, and sodium salts. Salts in the solid form may exist in more than one crystal structure, and may also be in the form of hydrates. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N′-dibenzylethylene-diamine, diethylamine, 2-diethylaminoethanol, 2-dimethylaminoethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like.

When the compound of the present invention is basic, salts may be prepared from pharmaceutically acceptable non-toxic acids, including inorganic and organic acids. Such acids include acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid, and the like. Particularly preferred are citric, hydrobromic, hydrochloric, maleic, phosphoric, sulfuric, fumaric, and tartaric acids. It will be understood that, as used herein, references to the compounds of Formula I are meant to also include the pharmaceutically acceptable salts.

Exemplifying the invention is the use of the compounds disclosed in the Examples and herein. Specific compounds within the present invention include a compound which selected from the group consisting of the compounds disclosed in the following Examples and pharmaceutically acceptable salts thereof and individual diastereomers thereof.

The subject compounds are useful in a method of potentiating metabotorpic glutamate receptor activity in a patient such as a mammal in need of such inhibition comprising the administration of an effective amount of the compound. The present invention is directed to the use of the compounds disclosed herein as potentiators of metabotorpic glutamate receptor activity. In addition to primates, especially humans, a variety of other mammals can be treated according to the method of the present invention.

The present invention is further directed to a method for the manufacture of a medicament for potentiating metabotorpic glutamate receptor activity in humans and animals comprising combining a compound of the present invention with a pharmaceutical carrier or diluent.

The subject treated in the present methods is generally a mammal, preferably a human being, male or female, in whom potentiation of metabotorpic glutamate receptor activity is desired. The term “therapeutically effective amount” means the amount of the subject compound that will elicit the biological or medical response of a tissue, system, animal or human that is being sought by the researcher, veterinarian, medical doctor or other clinician. It is recognized that one skilled in the art may affect the neurological and psychiatric disorders by treating a patient presently afflicted with the disorders or by prophylactically treating a patient afflicted with the disorders with an effective amount of the compound of the present invention.

As used herein, the terms “treatment” and “treating” refer to treatment of the noted conditions, to ameliorating or controlling all processes wherein there may be a slowing, interrupting, arresting, controlling, or stopping of the progression of the disorder but does not necessarily indicate a total elimination of all disorder symptoms, as well as the prevention or prophylactic therapy to retard the progression or reduce the risk of the noted conditions, particularly in a patient who is predisposed to such disease or disorder.

The term “composition” as used herein is intended to encompass a product comprising the specified ingredients in the specified amounts, as well as any product which results, directly or indirectly, from combination of the specified ingredients in the specified amounts. Such term in relation to pharmaceutical composition, is intended to encompass a product comprising the active ingredient(s), and the inert ingredient(s) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing a compound of the present invention and a pharmaceutically acceptable carrier. By “pharmaceutically acceptable” it is meant the carrier, diluent or excipient must be compatible with the other ingredients of the formulation and not deleterious to the recipient thereof.

The terms “administration of” and or “administering a” compound should be understood to mean providing a compound of the invention or a prodrug of a compound of the invention to the individual in need of treatment.

The utility of the compounds in accordance with the present invention as inhibitors of metabotropic glutamate receptor activity, in particular mGluR2 activity, may be demonstrated by methodology known in the art. Inhibition constants are determined as follows. The compounds of the present invention were tested in a [³⁵S]-GTPγS assay. The stimulation of [³⁵S]-GTPγS binding is a common functional assay to monitor Gαi-coupled receptor in native and recombinant receptor membrane preparation. Membrane from cells stably expressing hmGlu2 CHO-K1 (50 μg) were incubated in a 96 well plate for 1 hour in the presence of GTPγS³⁵ (0.05 nM), GDP (5 μM) and compounds. The reaction was stopped by rapid filtration over Unifilter GF/B plate (Packard, Bioscience, Meriden Conn.) using a 96-well cell harvester (Brandel Gaithersburg, Md.). The filter plates were counted using Topcount counter (Packard, Bioscience, Meriden Conn., USA). When compounds were evaluated as potentiator they were tested in the presence of glutamate (1 μl). The activation (agonist) or the potentiation of glutamate (potentiator) curves were fitted with a four parameters logistic equation giving EC₅₀ and Hill coefficient using the iterative non linear curve fitting software GraphPad (San Diego Calif., USA).

In particular, the compounds of the following examples had activity in potentiating the mGluR2 receptor in the aforementioned assays, generally with an EC₅₀ of less than about 10 μM. Preferred compounds within the present invention had activity in potentiating the mGluR2 receptor in the aforementioned assays with an EC₅₀ of less than about 1 μM. Such a result is indicative of the intrinsic activity of the compounds in use as potentiators of mGluR2 receptor activity.

Metabotropic glutamate receptors including the mGluR2 receptor have been implicated in a wide range of biological functions. This has suggested a potential role for these receptors in a variety of disease processes in humans or other species.

The compounds of the present invention have utility in treating, preventing, ameliorating, controlling or reducing the risk of a variety of neurological and psychiatric disorders associated with glutamate dysfunction, including one or more of the following conditions or diseases: acute neurological and psychiatric disorders such as cerebral deficits subsequent to cardiac bypass surgery and grafting, stroke, cerebral ischemia, spinal cord trauma, head trauma, perinatal hypoxia, cardiac arrest, hypoglycemic neuronal damage, dementia (including AIDS-induced dementia), Alzheimer's disease, Huntington's Chorea, amyotrophic lateral sclerosis, ocular damage, retinopathy, cognitive disorders, idiopathic and drug-induced Parkinson's disease, muscular spasms and disorders associated with muscular spasticity including tremors, epilepsy, convulsions, migraine (including migraine headache), urinary incontinence, substance tolerance, substance withdrawal (including, substances such as opiates, nicotine, tobacco products, alcohol, benzodiazepines, cocaine, sedatives, hypnotics, etc.), psychosis, schizophrenia, anxiety (including generalized anxiety disorder, panic disorder, and obsessive compulsive disorder), mood disorders (including depression, mania, bipolar disorders), trigeminal neuralgia, hearing loss, tinnitus, macular degeneration of the eye, emesis, brain edema, pain (including acute and chronic pain states, severe pain, intractable pain, neuropathic pain, and post-traumatic pain), tardive dyskinesia, sleep disorders (including narcolepsy), attention deficit/hyperactivity disorder, and conduct disorder.

Of the disorders above, the treatment of migraine, anxiety, schizophrenia, and epilepsy are of particular importance. In a preferred embodiment the present invention provides a method for treating migraine, comprising:

administering to a patient in need thereof an effective amount of a compound of formula I. In another preferred embodiment the present invention provides a method for preventing or treating anxiety, comprising: administering to a patient in need thereof an effective amount of a compound of formula I. Particularly preferred anxiety disorders are generalized anxiety disorder, panic disorder, and obsessive compulsive disorder. In another preferred embodiment the present invention provides a method for treating schizophrenia, comprising: administering to a patient in need thereof an effective amount of a compound of formula I. In yet another preferred embodiment the present invention provides a method for treating epilepsy, comprising: administering to a patient in need thereof an effective amount of a compound of formula I.

Of the neurological and psychiatric disorders associated with glutamate dysfunction which are treated according to the present invention, the treatment of migraine, anxiety, schizophrenia, and epilepsy are particularly preferred. Particularly preferred anxiety disorders are generalized anxiety disorder, panic disorder, and obsessive compulsive disorder.

Thus, in a preferred embodiment the present invention provides a method for treating migraine, comprising: administering to a patient in need thereof an effective amount of a compound of formula I or a pharmaceutical composition thereof. In one of the available sources of diagnostic tools, Dorland's Medical Dictionary (23'd Ed., 1982, W. B. Saunders Company, Philidelphia, Pa.), migraine is defined as a symptom complex of periodic headaches, usually temporal and unilateral, often with irritability, nausea, vomiting, constipation or diarrhea, and photophobia. As used herein the term “migraine” includes these periodic headaches, both temporal and unilateral, the associated irritability, nausea, vomiting, constipation or diarrhea, photophobia, and other associated symptoms. The skilled artisan will recognize that there are alternative nomenclatures, nosologies, and classification systems for neurological and psychiatric disorders, including migraine, and that these systems evolve with medical scientific progress.

In another preferred embodiment the present invention provides a method for treating anxiety, comprising: administering to a patient in need thereof an effective amount of a compound of formula I or a pharmaceutical composition thereof. At present, the fourth edition of the Diagnostic and Statistical Manual of Mental Disorders (DSM-IV) (1994, American Psychiatric Association, Washington, D.C.), provides a diagnostic tool including anxiety and related disorders. These include: panic disorder with or without agoraphobia, agoraphobia without history of panic disorder, specific phobia, social phobia, obsessive-compulsive disorder, post-traumatic stress disorder, acute stress disorder, generalized anxiety disorder, anxiety disorder due to a general medical condition, substance-induced anxiety disorder and anxiety disorder not otherwise specified. As used herein the term “anxiety” includes treatment of those anxiety disorders and related disorder as described in the DSM-IV. The skilled artisan will recognize that there are alternative nomenclatures, nosologies, and classification systems for neurological and psychiatric disorders, and particular anxiety, and that these systems evolve with medical scientific progress. Thus, the term “anxiety” is intended to include like disorders that are described in other diagnostic sources.

In another preferred embodiment the present invention provides a method for treating depression, comprising: administering to a patient in need thereof an effective amount of a compound of formula I or a pharmaceutical composition thereof. At present, the fourth edition of the Diagnostic and Statistical Manual of Mental Disorders (DSM-IV) (1994, American Psychiatric Association, Washington, D.C.), provides a diagnostic tool including depression and related disorders. Depressive disorders include, for example, single episodic or recurrent major depressive disorders, and dysthymic disorders, depressive neurosis, and neurotic depression; melancholic depression including anorexia, weight loss, insomnia and early morning waking, and psychomotor retardation; atypical depression (or reactive depression) including increased appetite, hypersomnia, psychomotor agitation or irritability, anxiety and phobias; seasonal affective disorder; or bipolar disorders or manic depression, for example, bipolar I disorder, bipolar II disorder and cyclothymic disorder. As used herein the term “depression” includes treatment of those depression disorders and related disorder as described in the DSM-IV.

In another preferred embodiment the present invention provides a method for treating epilepsy, comprising: administering to a patient in need thereof an effective amount of a compound of formula I or a pharmaceutical composition thereof. At present, there are several types and subtypes of seizures associated with epilepsy, including idiopathic, symptomatic, and cryptogenic. These epileptic seizures can be focal (partial) or generalized. They can also be simple or complex. Epilepsy is described in the art, such as Epilepsy: A comprehensive textbook. Ed. by Jerome Engel, Jr. and Timothy A. Pedley. (Uppincott-Raven, Philadelphia, 1997). At present, the International Classification of Diseases, Ninth Revision, (ICD-9) provides a diagnostic tool including epilepsy and related disorders. These include: generalized nonconvulsive epilepsy, generalized convulsive epilepsy, petit mal status epilepticus, grand mal status epilepticus, partial epilepsy with impairment of consciousness, partial epilepsy without impairment of consciousness, infantile spasms, epilepsy partialis continua, other forms of epilepsy, epilepsy, unspecified, NOS. As used herein the term “epilepsy” includes these all types and subtypes. The skilled artisan will recognize that there are alternative nomenclatures, nosologies, and classification systems for neurological and psychiatric disorders, including epilepsy, and that these systems evolve with medical scientific progress.

The subject compounds are further useful in a method for the prevention, treatment, control, amelioration, or reducation of risk of the diseases, disorders and conditions noted herein.

The subject compounds are further useful in a method for the prevention, treatment, control, amelioration, or reduction of risk of the aforementioned diseases, disorders and conditions in combination with other agents, including an mGluR agonist.

The term “potentiated amount” refers to an amount of an mGluR agonist, that is, the dosage of agonist which is effective in treating the neurological and psychiatric disorders described herein when administered in combination with an effective amount of a compound of the present invention. A potentiated amount is expected to be less than the amount that is required to provided the same effect when the mGluR agonist is administered without an effective amount of a compound of the present invention.

A potentiated amount can be readily determined by the attending diagnostician, as one skilled in the art, by the use of conventional techniques and by observing results obtained under analogous circumstances. In determining a potentiated amount, the dose of an mGluR agonist to be administered in combination with a compound of formula I, a number of factors are considered by the attending diagnostician, including, but not limited to: the mGluR agonist selected to be administered, including its potency and selectivity; the compound of formula I to be coadministered; the species of mammal; its size, age, and general health; the specific disorder involved; the degree of involvement or the severity of the disorder; the response of the individual patient; the modes of administration; the bioavailability characteristics of the preparations administered; the dose regimens selected; the use of other concomitant medication; and other relevant circumstances.

A potentiated amount of an mGluR agonist to be administered in combination with an effective amount of a compound of formula I is expected to vary from about 0.1 milligram per kilogram of body weight per day (mg/kg/day) to about 100 mg/kg/day and is expected to be less than the amount that is required to provided the same effect when administered without an effective amount of a compound of formula 1. Preferred amounts of a co-administered mGlu agonist are able to be determined by one skilled in the art.

The compounds of the present invention may be used in combination with one or more other drugs in the treatment, prevention, control, amelioration, or reduction of risk of diseases or conditions for which compounds of Formula I or the other drugs may have utility, where the combination of the drugs together are safer or more effective than either drug alone. Such other drug(s) may be administered, by a route and in an amount commonly used therefor, contemporaneously or sequentially with a compound of Formula I. When a compound of Formula I is used contemporaneously with one or more other drugs, a pharmaceutical composition in unit dosage form containing such other drugs and the compound of Formula I is preferred. However, the combination therapy may also includes therapies in which the compound of Formula I and one or more other drugs are administered on different overlapping schedules. It is also contemplated that when used in combination with one or more other active ingredients, the compounds of the present invention and the other active ingredients may be used in lower doses than when each is used singly. Accordingly, the pharmaceutical compositions of the present invention include those that contain one or more other active ingredients, in addition to a compound of Formula I.

The above combinations include combinations of a compound of the present invention not only with one other active compound, but also with two or more other active compounds.

Likewise, compounds of the present invention may be used in combination with other drugs that are used in the prevention, treatment, control, amelioration, or reduction of risk of the diseases or conditions for which compounds of the present invention are useful. Such other drugs may be administered, by a route and in an amount commonly used therefor, contemporaneously or sequentially with a compound of the present invention. When a compound of the present invention is used contemporaneously with one or more other drugs, a pharmaceutical composition containing such other drugs in addition to the compound of the present invention is preferred. Accordingly, the pharmaceutical compositions of the present invention include those that also contain one or more other active ingredients, in addition to a compound of the present invention.

The weight ratio of the compound of the compound of the present invention to the second active ingredient may be varied and will depend upon the effective dose of each ingredient. Generally, an effective dose of each will be used. Thus, for example, when a compound of the present invention is combined with another agent, the weight ratio of the compound of the present invention to the other agent will generally range from about 1000:1 to about 1:1000, preferably about 200:1 to about 1:200. Combinations of a compound of the present invention and other active ingredients will generally also be within the aforementioned range, but in each case, an effective dose of each active ingredient should be used.

In such combinations the compound of the present invention and other active agents may be administered separately or in conjunction. In addition, the administration of one element may be prior to, concurrent to, or subsequent to the administration of other agent(s).

The compounds of the present invention may be administered by oral, parenteral (e.g., intramuscular, intraperitoneal, intravenous, ICV, intracisternal injection or infusion, subcutaneous injection, or implant), by inhalation spray, nasal, vaginal, rectal, sublingual, or topical routes of administration and may be formulated, alone or together, in suitable dosage unit formulations containing conventional non-toxic pharmaceutically acceptable carriers, adjuvants and vehicles appropriate for each route of administration. In addition to the treatment of warm-blooded animals such as mice, rats, horses, cattle, sheep, dogs, cats, monkeys, etc., the compounds of the invention are effective for use in humans.

The pharmaceutical compositions for the administration of the compounds of this invention may conveniently be presented in dosage unit form and may be prepared by any of the methods well known in the art of pharmacy. All methods include the step of bringing the active ingredient into association with the carrier which constitutes one or more accessory ingredients. In general, the pharmaceutical compositions are prepared by uniformly and intimately bringing the active ingredient into association with a liquid carrier or a finely divided solid carrier or both, and then, if necessary, shaping the product into the desired formulation. In the pharmaceutical composition the active object compound is included in an amount sufficient to produce the desired effect upon the process or condition of diseases. As used herein, the term “composition” is intended to encompass a product comprising the specified ingredients in the specified amounts, as well as any product which results, directly or indirectly, from combination of the specified ingredients in the specified amounts.

The pharmaceutical compositions containing the active ingredient may be in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated by the techniques described in the U.S. Pat. Nos. 4,256,108; 4,166,452; and 4,265,874 to form osmotic therapeutic tablets for control release.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin, or olive oil.

Aqueous suspensions contain the active materials in admixture with excipients suitable for the manufacture of aqueous suspensions. Such excipients are suspending agents, for example sodium carboxymethylcellulose, methylcellulose, hydroxy-propylmethylcellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl, p-hydroxybenzoate, one or more coloring agents, one or more flavoring agents, and one or more sweetening agents, such as sucrose or saccharin.

Oily suspensions may be formulated by suspending the active ingredient in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

The pharmaceutical compositions of the invention may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening and flavoring agents.

Syrups and elixirs may be formulated with sweetening agents, for example glycerol, propylene glycol, sorbitol or sucrose. Such formulations may also contain a demulcent, a preservative and flavoring and coloring agents.

The pharmaceutical compositions may be in the form of a sterile injectable aqueous or oleagenous suspension. This suspension may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, for example as a solution in 1,3-butane diol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The compounds of the present invention may also be administered in the form of suppositories for rectal administration of the drug. These compositions can be prepared by mixing the drug with a suitable non-irritating excipient which is solid at ordinary temperatures but liquid at the rectal temperature and will therefore melt in the rectum to release the drug. Such materials are cocoa butter and polyethylene glycols.

For topical use, creams, ointments, jellies, solutions or suspensions, etc., containing the compounds of The present invention are employed. (For purposes of this application, topical application shall include mouth washes and gargles.)

The pharmaceutical composition and method of the present invention may further comprise other therapeutically active compounds as noted herein which are usually applied in the treatment of the above mentioned pathological conditions.

In the treatment, prevention, control, amelioration, or reduction of risk of conditions which require potentiation of metabotorpic glutamate receptor activity an appropriate dosage level will generally be about 0.01 to 500 mg per kg patient body weight per day which can be administered in single or multiple doses. Preferably, the dosage level will be about 0.1 to about 250 mg/kg per day; more preferably about 0.5 to about 100 mg/kg per day. A suitable dosage level may be about 0.01 to 250 mg/kg per day, about 0.05 to 100 mg/kg per day, or about 0.1 to 50 mg/kg per day. Within this range the dosage may be 0.05 to 0.5, 0.5 to 5 or 5 to 50 mg/kg per day. For oral administration, the compositions are preferably provided in the form of tablets containing 1.0 to 1000 milligrams of the active ingredient, particularly 1.0, 5.0, 10.0, 15.0. 20.0, 25.0, 50.0, 75.0, 100.0, 150.0, 200.0, 250.0, 300.0, 400.0, 500.0, 600.0, 750.0, 800.0, 900.0, and 1000.0 milligrams of the active ingredient for the symptomatic adjustment of the dosage to the patient to be treated. The compounds may be administered on a regimen of 1 to 4 times per day, preferably once or twice per day.

When treating, preventing, controlling, ameliorating, or reducing the risk of neurological and psychiatric disorders associated with glutamate dysfunction or other diseases for which compounds of the present invention are indicated, generally satisfactory results are obtained when the compounds of the present invention are administered at a daily dosage of from about 0.1 milligram to about 100 milligram per kilogram of animal body weight, preferably given as a single daily dose or in divided doses two to six times a day, or in sustained release form. For most large mammals, the total daily dosage is from about 1.0 milligrams to about 1000 milligrams, preferably from about 1 milligrams to about 50 milligrams. In the case of a 70 kg adult human, the total daily dose will generally be from about 7 milligrams to about 350 milligrams. This dosage regimen may be adjusted to provide the optimal therapeutic response.

It will be understood, however, that the specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

Several methods for preparing the compounds of this invention are illustrated in the following Schemes and Examples. Starting materials are made according to procedures known in the art or as illustrated herein.

The compounds of the present invention can be prepared in a variety of fashions.

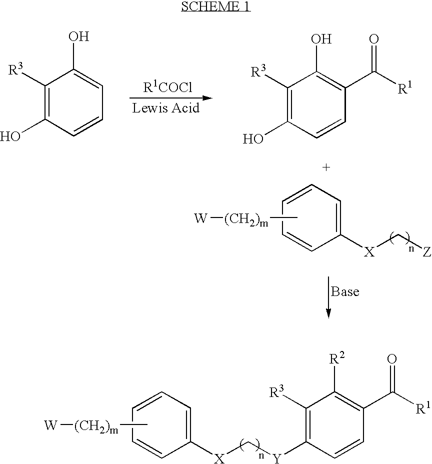

An appropriately substituted acetophenone precursor can be prepared via an acylation followed by an alkylation as illustrated in Scheme 1. A substituted 1,3-bisphenol (either purchased commercially or prepared using techniques well known in the art) is reacted with an acid chloride in the presence of a Lewis acid, such as aluminum trichloride, tin tetrachloride and titanium tetrachloride can be used in solvents such as dichloromethane and nitrobenzene. The reaction generally proceeds by allowing the reaction to warm from 0° C. to ambient temperature over a period of several hours, and then maintained at ambient temperature for several more hours. The product from the reaction can be isolated and purified employing standard techniques such as solvent extraction, chromatography, crystallization, distillation and the like.

The product obtained is then alkylated with variously substituted aryl compounds.

These aryl compounds contain linkers with a suitable leaving group (wherein Z is halide, triflate, tosylate, mesylate and the like) and are reacted in the presence of a base (potassium carbonate, sodium hydroxide, and the like) in a suitable solvent (acetone, tetrahydrofuran, dimethoxyethane, etc.). The reaction is generally run at ambient temperature to 45° C. for a period of 4 to 24 hours. The product from the reaction can be isolated and purified employing standard techniques such as solvent extraction, chromatography, crystallization, distillation and the like.

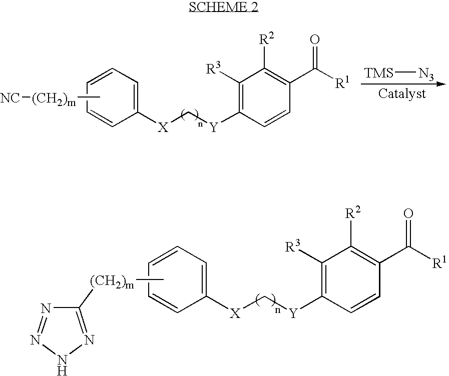

The alkylated compounds (when W=nitrile) can then be converted into tetrazoles as shown in Scheme 2. The nitrile derivative is reacted with trimethylsilyl azide in the presence of a catalyst such as dibutyltin oxide in a suitable solvent (benzene, toluene, mesitylene and the like) at an appropriate temperature, usually 110° C. for a period of 8-16 hours. The product from the reaction can be isolated and purified employing standard techniques such as solvent extraction, chromatography, crystallization, distillation and the like.

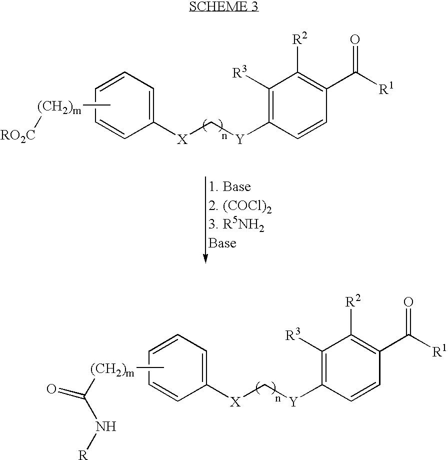

The alkylated compounds (when W=ester) can also be converted into carboxylic acids, amides, sulfonamides and imides as shown in Scheme 3. The ester derivative is first hydrolyzed in the presence of a suitable base (lithium hydroxide, sodium hydroxide and the like) in a solvent such as water/dioxane of water/tetrahydrofuran to provide the corresponding carboxylic acid. The reaction is generally run at ambient temperature for a period of 1-16 hours. The carboxylic acid can be further reacted by first converting it to the acid chloride via reaction with oxalyl chloride (or other reagents such as thionyl chloride) in a suitable solvent such as dichloromethane. This acid chloride can then be further reacted with a variety of nitrogen compounds such as amides and sulfonamides in the presence of a base such as sodium hydride or lithium diisopropyl amide in a suitable solvent such as tetrahydrofuran to give the desired compound. The reaction is generally run at temperatures from −78 to 0° C. for a period of 4-12 hours. The product from the reaction can be isolated and purified employing standard techniques such as solvent extraction, chromatography, crystallization, distillation and the like.

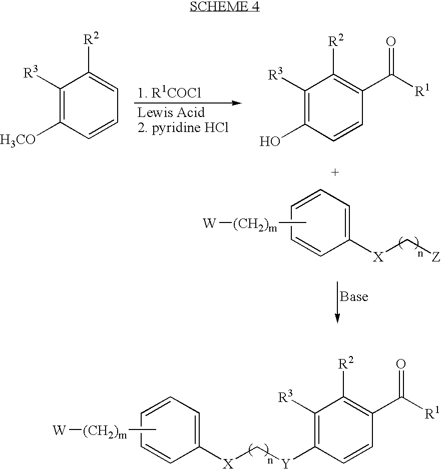

In a similar manner as in Scheme 1, a variety of substituted acetophenones can be synthesized as shown in Scheme 4. A 2,3-substituted anisole (either purchased commercially or prepared using techniques well known in the art) is reacted with an acid chloride in the presence of a lewis acid such as aluminum trichloride, tin tetrachloride and titanium tetrachloride in solvents such as dichloromethane and nitrobenzene. The reaction generally proceeds by allowing the reaction to warm from 0° C. to ambient temperature over a period of several hours, and then maintained at ambient temperature for several more hours. The methyl group of the anisole is then removed using a suitable reagent such as pyridine hydrochloride as a melt at temperatures of 150-175° C. The product from the reaction can be isolated and purified employing standard techniques such as solvent extraction, chromatography, crystallization, distillation and the like.

The product obtained is then alkylated with variously substituted aryl compounds. These aryl compounds contain linkers with a suitable leaving group (Z is halide, triflate, tosylate, mesylate and the like) and are reacted in the presence of a base (potassium carbonate, sodium hydroxide, and the like) in a suitable solvent (acetone, tetrahydrofuran, dimethoxyethane, etc.). The reaction is generally run at ambient temperature to 45° C. for a period of 4 to 24 hours. The product from the reaction can be isolated and purified employing standard techniques such as solvent extraction, chromatography, crystallization, distillation and the like.

The alkylated compounds can then be converted into tetrazoles (when W=nitrile) as or carboxylic acids, amides, sulfonamides and imides (when W=ester) via methods essentially as outlined in Schemes 2 and 3.

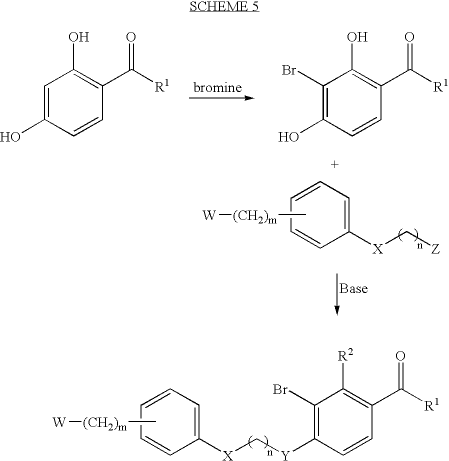

Brominated and other halogenated analogs can also be prepared as illustrated in Scheme 5. As shown, variously substituted 2,4-dihydroxy acetophenones can be treated with bromine in solvents such as ethanol to give brominated precursors, which can then be further reacted as described above in Schemes 3-5 to give the target compounds. Additional compounds may be prepared by reaction of the phenyl bromide.

In some cases the final product may be further modified, for example, by manipulation of substituents. These manipulations may include, but are not limited to, reduction, oxidation, alkylation, acylation, and hydrolysis reactions which are commonly known to those skilled in the art. In some cases the order of carrying out the foregoing reaction schemes may be varied to facilitate the reaction or to avoid unwanted reaction products. The following examples are provided so that the invention might be more fully understood. These examples are illustrative only and should not be construed as limiting the invention in any way.

### EXAMPLE 1

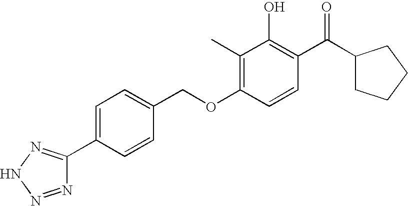

### Cyclopentyl-{2-hydroxy-3-methyl-4-[4-(2H-tetrazol-5-yl)-benzyloxy]-phenyl}-methanone

Cyclopentyl carbonyl chloride (690 mg, 0.63 mL, 5.2 mmol) was added to a stirred solution of 2-methylresorcinol (500 mg, 4.0 mmol) and aluminum trichloride (693 mg, 5.2 mmol) in dichloromethane (20 mL) at 0° C. The reaction was allowed to warm to rt, then stirred for 16 hr. It was then quenched by addition of 1N aqueous HCl. The organic layer was separated, dried over MgSO₄ and then concentrated in vacuo to give a residue that was purified via column chromatography on silica gel (eluting 5-50% ethyl acetate/hexanes) to give 370 mg (42%) of cyclopentyl-(2,4-dihydroxy-3-methyl-phenyl)-methanone as a colorless oil. Potassium carbonate (188 mg, 1.36 mmol) was added to a stirred solution of cyclopentyl-(2,4-dihydroxy-3-methyl-phenyl)-methanone (150 mg, 0.68 mmol) and 4-cyanobenzylbromide (160 mg, 0.82 mmol) in acetone (10 mL) at 45° C. The reaction mixture was stirred for 16 hr, then the acetone was removed in vacuo. The residue was then mixed with dichloromethane (50 mL) and water (50 mL). The organic layer was separated, dried over MgSO₄ and then concentrated in vacuo to give a residue that was purified via column chromatography on silica gel (eluting 15-50% ethyl acetate/hexanes) to give 193 mg (85%) of 4-(4-Cyclopentanecarbonyl-3-hydroxy-2-methyl-phenoxymethyl)-benzonitrile as a white solid. 4-(4-Cyclopentanecarbonyl-3-hydroxy-2-methyl-phenoxymethyl)-benzonitrile (100 mg, 0.30 mmol), trimethylsilylazide (69 mg, 0.08 mL, 0.60 mmol) and dibutyltin oxide (11 mg, 0.045 mmol) were dissolved in toluene (8 mL) and heated to reflux for 16 hr. The reaction mixture was then cooled to rt and applied directly to a silica gel column (eluting first with 20% ethyl acetate/hexanes followed by 10% MeOH/dichloromethane) to give cyclopentyl-{2-hydroxy-3-methyl-4-[4-(2!-tetrazol-5-yl)-benzyloxy]-phenyl}-methanone as a white solid. ¹H NMR(DMSO-d₆, 500 MHz), δ 13.1 (s, 1H), 8.1 (d, 2H), 7.9 (d, 1H), 7.6 (d, 2H), 6.7 (d, 1H), 5.4 (s, 2H), 3.9 (quint, 1H), 3.2 (s, 1H), 2.1 (s, 3H), 1.91-1.87 (m, 2H), 1.77-1.72 (m, 2H), 1.67-1.62 (m, 4H). MS (ESI): 379 (M+H)⁺.

### EXAMPLE 2

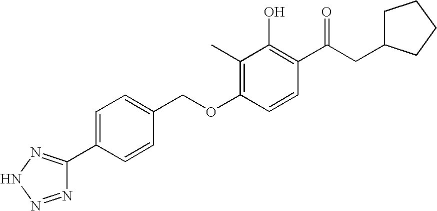

### 2-Cyclopentyl-1-{2-hydroxy-3-methyl-4-[4-(2H-tetrazol-5-yl)-benzyloxy]-phenyl}-ethanone

Cyclopentylacetyl chloride (1.53 g, 1.4 mL, 10.4 mmol) was added to a stirred solution of 2-methylresorcinol (1 g, 8.0 mmol) and aluminum trichloride (1.39 g, 10.4 mmol) in dichloromethane (40 mL) at 0° C. The reaction was allowed to warm to rt, then stirred for 16 hr. It was then quenched by addition of 1N aqueous HCl. The organic layer was separated, dried over MgSO₄ and then concentrated in vacuo to give a residue that was purified via column chromatography on silica gel (eluting 5-50% ethyl acetate/hexanes) to give 1.16 g (62%) of 2-cyclopentyl-1-(2,4-dihydroxy-3-methyl-phenyl)-ethanone as a white solid.

Potassium carbonate (580 mg, 4.2 mmol) was added to a stirred solution of 2-cyclopentyl-1-(2,4-dihydroxy-3-methyl-phenyl)-ethanone (500 mg, 2.1 mmol) and 4-cyanobenzylbromide (500 mg, 2.6 mmol) in acetone (40 mL) at 45° C. The reaction mixture was stirred for 16 hr, then the acetone was removed in vacuo. The residue was then mixed with dichloromethane (100 mL) and water (100 mL). The organic layer was separated, dried over MgSO₄ and then concentrated in vacuo to give a residue that was purified via column chromatography on silica gel (eluting 10-50% ethyl acetate/hexanes) to give 459 mg (61%) of 4-[4-(2-Cyclopentyl-acetyl)-3-hydroxy-2-methyl-phenoxymethyl]-benzonitrile as a white solid. 4-[4-(2-Cyclopentyl-acetyl)-3-hydroxy-2-methyl-phenoxymethyl]-benzonitrile (300 mg, 0.86 mmol), trimethylsilylazide (198 mg, 0.23 mL, 1.72 mmol) and dibutyltin oxide (32 mg, 0.13 mmol) were dissolved in toluene (15 mL) and heated to reflux for 16 hr. The reaction mixture was then cooled to rt and applied directly to a silica gel column (eluting first with 20% ethyl acetate/hexanes followed by 10% MeOH/dichloromethane) to give 2-cyclopentyl-1-{2-hydroxy-3-methyl-4-[4-(2H-tetrazol-5-yl)-benzyloxy]-phenyl}-ethanone as a white solid. ¹H NMR (DMSO-d₆, 500 MHz), δ 13.02 (s, 1H), 8.07 (d, 2H), 7.87 (d, 1H), 7.65 (d, 2H), 6.73 (d, 1H), 5.35 (s, 2H), 3.17 (s, 1H), 3.00 (d, 2H), 2.25 (sept, 1H), 2.09 (s, 3H), 1.77-1.75 (m, 2H), 1.62-1.59 (m, 2H), 1.51-1.49 (m, 2H), 1.20-1.15 (m, 2H). MS (ESI): 393 (M+H)⁺.

### EXAMPLE 3

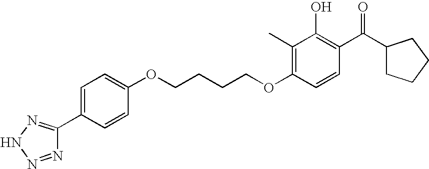

### Cyclopentyl-(2-hydroxy-3-methyl-4-{4-[4-(2H-tetrazol-5-yl)-phenoxy]-butoxy}-phenyl)-methanone

Potassium carbonate (188 mg, 1.36 mmol) was added to a stirred solution of cyclopentyl-(2,4-dihydroxy-3-methyl-phenyl)-methanone (150 mg, 0.68 mmol) and 4-(4-Bromo-butoxy)-benzonitrile (208 mg, 0.82 mmol) in acetone (10 mL) at 45° C. The reaction mixture was stirred for 16 hr, then the acetone was removed in vacuo. The residue was then mixed with dichloromethane (50 mL) and water (50 mL). The organic layer was separated, dried over MgSO₄ and then concentrated in vacuo to give a residue that was purified via column chromatography on silica gel (eluting 5-50% ethyl acetate/hexanes) to give 182 mg (68%) of 4-[4-(4-Cyclopentanecarbonyl-3-hydroxy-2-methyl-phenoxy)-butoxy]-benzonitrile as a white solid. 4-[4-(4-Cyclopentanecarbonyl-3-hydroxy-2-methyl-phenoxy)-butoxy]-benzonitrile (182 mg, 0.46 mmol), trimethylsilylazide (107 mg, 0.13 mL, 0.93 mmol) and dibutyltin oxide (17 mg, 0.069 mmol) were dissolved in toluene (10 mL) and heated to reflux for 16 hr. The reaction mixture was then cooled to rt and applied directly to a silica gel column (eluting first with 20% ethyl acetate/hexanes followed by 10% MeOH/dichloromethane) to give cyclopentyl-(2-hydroxy-3-methyl-4-{4-[4-(2H-tetrazol-5-yl)-phenoxy]-butoxy}-phenyl)-methanone as a white solid. ¹H NMR (DMSO-d₆, 500 MHz), δ 13.03 (s, 1H), 7.97 (d, 2H), 7.88 (d, 1H), 7.15 (d, 2H), 6.66 (d, 1H), 4.19-4.09 (m, 4H), 3.85 (quint, 1H), 2.00 (s, 3H), 1.94-1.88 (m, 6H), 1.78-1.74 (m, 2H), 1.66-1.50 (m, 4H). MS (ESI): 437 (M+H)⁺.

### EXAMPLE 4

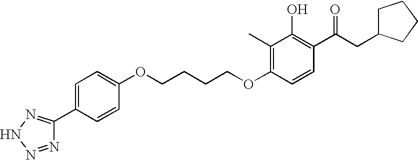

### 2-Cyclopentyl-1-(2-hydroxy-3-methyl-4-{4-[4-(2H-tetrazol-5-yl)-phenoxy]-butoxy}-phenyl)-ethanone

Potassium carbonate (580 mg, 4.2 mmol) was added to a stirred solution of 2-cyclopentyl-1-(2,4-dihydroxy-3-methyl-phenyl)-ethanone (500 mg, 2.1 mmol) and 4-(4-Bromo-butoxy)-benzonitrile (661 mg, 2.6 mmol) in acetone (40 mL) at 45° C. The reaction mixture was stirred for 16 hr, then the acetone was removed in vacuo. The residue was then mixed with dichloromethane (100 mL) and water (100 mL). The organic layer was separated, dried over MgSO₄ and then concentrated in vacuo to give a residue that was purified via column chromatography on silica gel (eluting 5-50% ethyl acetate/hexanes) to give 530 mg (62%) of 4-{4-[4-(2-Cyclopentyl-acetyl)-3-hydroxy-2-methyl-phenoxy]-butoxy}-benzonitrile as a white solid. 4-{4-[4-(2-Cyclopentyl-acetyl)-3-hydroxy-2-methyl-phenoxy]-butoxy}-benzonitrile (530 mg, 1.3 mmol), trimethylsilylazide (300 mg, 0.35 mL, 2.6 mmol) and dibutyltin oxide (49 mg, 0.20 mmol) were dissolved in toluene (20 mL) and heated to reflux for 16 hr. The reaction mixture was then cooled to rt and applied directly to a silica gel column (eluting first with 20% ethyl acetate/hexanes followed by 10% MeOH/dichloromethane) to give 2-cyclopentyl-1-(2-hydroxy-3-methyl-4-{4-[4-(2H-tetrazol-5-yl)-phenoxy]-butoxy}-phenyl)-ethanone as a white solid. ¹H NMR (DMSO-d₆, 500 MHz), δ 13.02 (s, 1H), 7.99 (d, 2H), 7.80 (d, 1H), 7.15 (d, 2H), 6.65 (d, 1H), 4.57-4.07 (m, 4H), 3.00 (d, 2H), 2.31-2.28 (m, 1H), 2.00 (s, 3H), 1.95-1.92 (m, 4H), 1.77-1.74 (m, 2H), 1.61-0.149 (m, 6H). MS (ESI): 451 (M+H)⁺.

### EXAMPLE 5

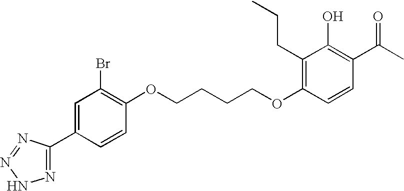

### 1-(4-{4-[2-Bromo-4-(2H-tetrazol-5-yl)-phenoxy]-butoxy}-2-hydroxy-3-propyl-phenyl)-ethanone

Potassium carbonate (690 mg, 5.0 mmol) was added to a stirred solution of 1-(2,4-Dihydroxy-3-propyl-phenyl)-ethanone (490 mg, 2.5 mmol) and 3-Bromo-4-(4-bromo-butoxy)-benzonitrile (1.00 g, 3.0 mmol) in acetone (40 mL) at 45° C. The reaction mixture was stirred for 16 hr, then the acetone was removed in vacuo. The residue was then mixed with dichloromethane (100 mL) and water (100 mL). The organic layer was separated, dried over MgSO₄ and then concentrated in vacuo to give a residue that was purified via column chromatography on silica gel (eluting 5-60% ethyl acetate/hexanes) to give 657 mg (59%) of 4-[4-(4-Acetyl-3-hydroxy-2-propyl-phenoxy)-butoxy]-3-bromo-benzonitrile as a white solid. 4-[4-(4-Acetyl-3-hydroxy-2-propyl-phenoxy)-butoxy]-3-bromo-benzonitrile (400 mg, 0.90 mmol), trimethylsilylazide (207 mg, 0.24 mL, 1.8 mmol) and dibutyltin oxide (34 mg, 0.135 mmol) were dissolved in toluene (15 mL) and heated to reflux for 16 hr. The reaction mixture was then cooled to rt and applied directly to a silica gel column (eluting first with 20% ethyl acetate/hexanes followed by 10% MeOH/dichloromethane) to give 1-(4-{4-[2-Bromo-4-(2H-tetrazol-5-yl)-phenoxy]-butoxy}-2-hydroxy-3-propyl-phenyl)-ethanone as a white solid. ¹H NMR (DMSO-d₆, 500 MHz), δ 12.85 (s, 1H), 8.20 (d, 1H), 8.00 (dd, 1H), 7.81 (d, 1H), 7.29 (d, 1H), 6.67 (d, 1H), 4.25-4.19 (m, 4H), 3.17 (s, 1H), 2.58 (s, 3H), 2.56-2.52 (m, 2H), 1.98-1.96 (m, 4H), 1.48-1.43 (m, 2H), 0.86 (t, 3H). MS (ESI): 489 (M+H)⁺.

### EXAMPLE 6

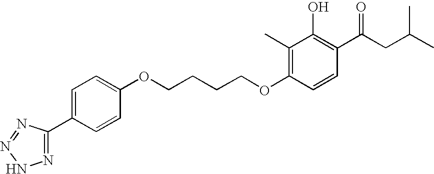

### 1-(2-Hydroxy-3-methyl-4-{4-[4-(2H-tetrazol-5-yl)-phenoxy]-butoxy}-phenyl)-3-methyl-butan-1-one

Isobutyryl chloride (1.25 g, 1.3 mL, 10.4 mmol) was added to a stirred solution of 2-methylresorcinol (1 g, 8.0 mmol) and aluminum trichloride (1.39 g, 10.4 mmol) in dichloromethane (40 mL) at 0° C. The reaction was allowed to warm to rt, then stirred for 16 hr. It was then quenched by addition of 1N aqueous HCl. The organic layer was separated, dried over MgSO₄ and then concentrated in vacuo to give a residue that was purified via column chromatography on silica gel (eluting 5-60% ethyl acetate/hexanes) to give 946 mg (57%) of 1-(2,4-Dihydroxy-3-methyl-phenyl)-3-methyl-butan-1-one as a white solid.

Potassium carbonate (398 mg, 2.88 mmol) was added to a stirred solution of 1-(2,4-Dihydroxy-3-methyl-phenyl)-3-methyl-butan-1-one (300 mg, 1.44 mmol) and 4-(4-Bromo-butoxy)-benzonitrile (403 mg, 1.58 mmol) in acetone (20 mL) at 45° C. The reaction mixture was stirred for 16 hr, then the acetone was removed in vacuo. The residue was then mixed with dichloromethane (100 mL) and water (100 mL). The organic layer was separated, dried over MgSO₄ and then concentrated in vacuo to give a residue that was purified via column chromatography on silica gel (eluting 5-50% ethyl acetate/hexanes) to give 378 mg (69%) of 4-{4-[3-Hydroxy-2-methyl-4-(3-methyl-butyryl)-phenoxy]-butoxy}-benzonitrile as a white solid. 4-{4-[3-Hydroxy-2-methyl-4-(3-methyl-butyryl)-phenoxy]-butoxy}-benzonitrile (257 mg, 0.67 mmol), trimethylsilylazide (155 mg, 0.18 mL, 1.3 mmol) and dibutyltin oxide (25 mg, 0.10 mmol) were dissolved in toluene (12 mL) and heated to reflux for 16 hr. The reaction mixture was then cooled to rt and applied directly to a silica gel column (eluting first with 20% ethyl acetate/hexanes followed by 10% MeOH/dichloromethane) to give 1-(2-Hydroxy-3-methyl-4-{4-[4-(2H-tetrazol-5-yl)-phenoxy]-butoxy}-phenyl)-3-methyl-butan-1-one as a white solid. ¹H NMR (DMSO-d₆, 500 MHz), δ 13.03 (s, 1H), 7.95 (d, 2H), 7.85 (d, 1H), 7.10 (d, 2H), 6.65 (d, 1H), 4.18-4.13 (m, 4H), 2.85 (d, 2H), 2.17-2.14 (m, 1H), 2.00 (s, 3H), 1.95-1.93 (m, 4H), 0.93 (d, 6H). MS (ESI): 425 (M+H)⁺.

### EXAMPLE 7

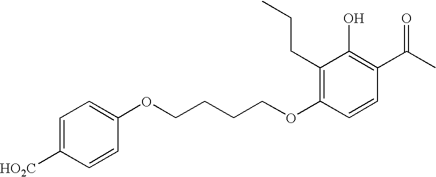

### 4[4-(4-Acetyl-3-hydroxy-2-propyl-phenoxy)-butoxy]-benzoic acid

Potassium carbonate (1.0 g, 7.7 mmol) was added to a stirred solution of 1-(2,4-dihydroxy-3-propyl-phenyl)-ethanone (1.0 g, 5.2 mmol) and 4-(4-bromo-butoxy)-benzoic acid methyl ester (1.6 g, 5.7 mmol) in acetone (52 ml) at 45° C. The reaction mixture was stirred for 18 hr, then the acetone was removed in vacuo. The residue was washed with brine (150 ml) and extracted with dichloromethane (180 ml). The organic layer was separated, dried over Na₂SO₄, filtered and then concentrated in vacuo to give a residue that was purified via column chromatography on silica gel (eluting 1-35% ethyl acetate/hexanes) to give 1.9 g (90%) of 4-[4-(4-acetyl-3-hydroxy-2-propyl-phenoxy)-butoxy]-benzoic acid methyl ester as an oil. 1.0 N Lithium hydroxide (22 ml) was added to a solution of 4-[4-(4-acetyl-3-hydroxy-2-propyl-phenoxy)-butoxy]-benzoic acid methyl ester (1.8 g, 4.5 mmol) in tetrahydrofuran (22 ml). The mixture was refluxed for 16 hr and then cooled to 0° C. 1.0 N HCl aqueous solution was added to mixture until pH 5. The mixture was washed with brine (2×60 ml) and extracted with ethyl acetate (2×70 ml). Organic extracts were dried over Na₂SO₄, filtered and concentrated in vacuo to give a brown solid. The crude solid was purified by column chromatography on silica gel (eluting first with 1-100% ethyl acetate/hexanes followed by 10-20% methanol/ethyl acetate) to give 4-[4-(4-acetyl-3-hydroxy-2-propyl-phenoxy)-butoxy]-benzoic acid as a tan solid. ¹H NMR (DMSO-d₆, 500 MHz), 12.84 (s, 1H), 12.61 (s, 1H), 7.89 (d, 2H), 7.82 (d, 1H), 7.03 (d, 2H), 6.67 (d, 1H), 4.16 (m, 4H), 2.64 (s, 3H), 2.57 (m, 2H), 1.99 (m, 4H), 1.49 (m, 2H), 0.87 (t, 3H). MS (ESI): 387 (M+H)⁺.

### EXAMPLE 8

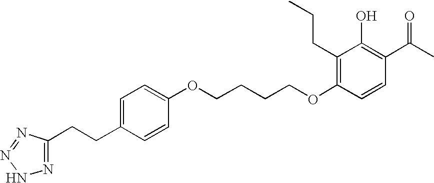

1-[2-Hydroxy-3-propyl-4-(4-(4-[2-(2H-tetrazol-5-yl)-ethyl)-phenoxy]-butoxy)-phenyl]-ethanone was prepared in a similar method as outlined for 1-(2-Hydroxy-3-propyl-4-{4-[4-(2H-tetrazol-5-yl)-phenoxy]-butoxy}-phenyl)-ethanone. MS (ESI): 439 (M+H)⁺.

### EXAMPLE 9

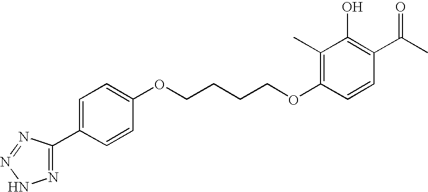

### 1-(2-Hydroxy-3-methyl-4-{4-[4-(2H-tetrazol-5-yl)-phenoxy]-butoxy}-phenyl)-ethanone

Potassium carbonate (1.577 g, 12.87 mmol) was added to a stirred solution of 4-(2-bromoethoxy)benzonitrile (1.57 g, 6.17 mmol) and 1-(2,4-dihydroxy-3-methyl-phenyl)-ethanone (0.85 g, 5.15 mmol) in acetone (50 mL). The reaction mixture was heated under reflux for 6 hours, filtered and then the acetone was removed in vacuo. The residue that was purified via column chromatography on silica gel (eluting 20-30% ethyl acetate/hexanes) to give 0.51 g (28.9%) of 4-[4-(4-acetyl-3-hydroxy-2-methyl-phenoxy)-butoxy]-benzonitrile as a white solid. MS (ESI): 340 (M+H)⁺. 4-[4-(4-Acetyl-3-hydroxy-2-methyl-phenoxy)-butoxy]-benzonitrile (500 mg, 1.47 mmol), trimethylsilylazide (424 mg, 0.49 mL, 3.68 mmol) and dibutyltin oxide (54 mg, 0.22 mmol) were dissolved in toluene (30 mL) and heated to reflux for 16 hr. Silica was added and the toluene was removed in vacuo. The crude material absorbed on silica was purified on the horizon biotage unit (eluting with 10% MeOH/chloroform) to give 1-(2-hydroxy-3-methyl-4-{4-[4-(2H-tetrazol-5-yl)-phenoxy)-butoxy}-phenyl)-ethanone as a white solid. ¹H NMR (DMSO-d₆, 500 MHz), δ 12.84 (s, 1H), 7.95 (d, 2H), 7.80 (d, 1H), 7.14 (d, 2H), 6.66 (d, 1H), 4.17-4.14 (m, 4H), 2.62 (s, 3H), 2.50-2.45 (m, 4H), 1.99 (s, 3H), 1.93-1.86 (m, 4H).

### EXAMPLE 10

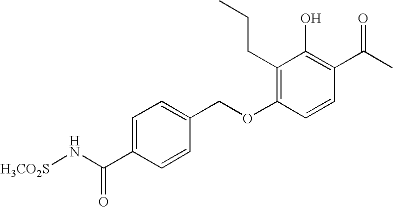

### N-[4-(4-Acetyl-3-hydroxy-2-propyl-phenoxymethyl)-benzoyl]-methanesulfonamide

Potassium carbonate (2.2 g, 15.9 mmol) was added to a stirred solution of 1-(2,4-dihydroxy-3-propyl-phenyl)-ethanone (2.0 g, 10.3 mmol) and methyl-4-(bromomethyl)-benzoate (2.6 g, 11.3 mmol) in acetone (100 ml) at 45° C. The reaction mixture was stirred for four hours and concentrated in vacuo. The resulting oil was washed with brine (2×80 ml) and extracted with dichloromethane (2×100 ml). The combined organic extracts were dried over Na₂SO₄, filtered and concentrated in vacuo to give a yellow solid. The solid was purified by column chromatography on silica gel (eluting 10-90% ethyl acetate/hexanes) to give 4-(4-acetyl-3-hydroxy-2-propyl-phenoxymethyl)-benzoic acid methyl ester as a white solid (3.4 g, 98%). A mixture of 4-(4-acetyl-3-hydroxy-2-propyl-phenoxymethyl)-benzoic acid methyl ester (3.4 g, 10.0 mmol), 1.0 N lithium hydroxide aqueous solution (50 ml) and tetrahydrofuran (50 ml) was heated to 75° C. for 18 hr and then cooled to rt. The reaction mixture was concentrated in vacuo and acidified to pH 5 with 6.0 N HCl aqueous solution. The mixture was washed with brine (200 ml) and extracted with ethyl acetate (2×100 ml). The organic layers were dried over Na₂SO₄, filtered and concentrated in vacuo to give 4-(4-acetyl-3-hydroxy-2-propyl-phenoxymethyl)-benzoic acid as a yellow solid (3.2 g, 100%). Freshly distilled oxalyl chloride (0.44 ml, 5.0 mmol) was added to a stirred solution of 4-(4-acetyl-3-hydroxy-2-propyl-phenoxymethyl)-benzoic acid (328 mg, 1.0 mmol) in dichloromethane (25 ml) at rt under nitrogen. Dimethylformamide (0.05 ml) was slowly added to the mixture until bubbling ceased. Mixture was allowed to stir until no starting material was observed by tlc and then concentrated in vacuo to give the acid chloride as a yellow solid. The acid chloride was dissolved in tetrahydrofuran (7.5 ml) and added in one portion to a cooled stirred solution of sodium hydride (120 mg, 3.0 mmol), methane sulfonamide (238 mg, 2.5 mmol) and tetrahydrofuran (5.0 ml) at 0° C. The reaction mixture was allowed to warm to rt overnight and then carefully quenched with water. The solution was washed with brine (2×30 ml) and extracted with ethyl acetate (2×60 ml), filtered and concentrated in vacuo to give an oil. The oil was purified by column chromatography (eluting first with 50-100% ethyl acetate/hexanes followed by 5-20% methanol/ethyl acetate) to give N-[4-(4-acetyl-3-hydroxy-2-propyl-phenoxymethyl)-benzoyl]-methanesulfonamide as a yellow foam. ¹H NMR (MSO-d₆, 500 MHz) 12.86 (s, 1H), 7.96 (d, 2H), 7.82 (d, 1H), 7.42 (d, 2H), 6.73 (d, 1H), 5.28 (s, 2H), 2.90 (s, 3H), 2.61 (m, 2H), 2.58 (s, 3H), 1.49 (m, 2H), 0.91 (t, 3H). MS(ESI): 406 (M+H)⁺.

### EXAMPLE 11

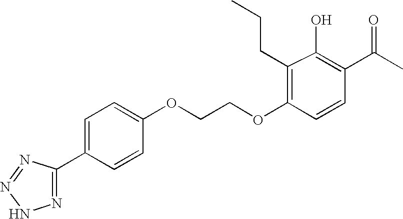

### 1-(2-Hydroxy-3-propyl-4-{2-[4-(1H-tetraazol-5-yl)phenoxy]ethoxy}phenyl)ethanone

To a stirred solution of 4-(2-bromoethoxy)benzonitrile (1.0 g, 4.4 mmol) in acetone (50 ml), was added K₂CO₃ (1.2 g, 8.8 mmol) and 2′4′-dihydroxy-3′-propylacetophenone (1.3 g, 6.6 mmol). The reaction mixture was allowed to stir at 60° C. for 18 hours, cooled to ambient temperature and filtered. The filtrate was concentrated in vacuo and the crude material was purified on a Biotage horizon system (eluting 5%-30% ethyl acetate/hexanes) to afford 4-[2-(4-acetyl-3-hydroxy-2-propylphenoxy)ethoxy]benzonitrile (0.7 g, 47%) as white solid. MS (ESI) 340(M++1).

To a degassed solution of 4-[2-(4-acetyl-3-hydroxy-2-propylphenoxy)ethoxy]benzonitrile (230 mg, 0.67 mmol) in toluene (3 ml) was added azidotrimethylsilane (310 mg, 2.7 mmol) and di-n-butyltinoxide (30 mg, 0.1 mmol). The reaction mixture was stirred at 110° C. for 20 hours, cooled to ambient temperature and concentrated under reduced pressure. The crude material was purified by semi-preparative HPLC to afford 1-(2-hydroxy-3-propyl-4-{2-[4-(1H-tetraazol-5-yl)phenoxy]ethoxylphenyl)-ethanone. ¹H NMR (CD₃OD, 500 MHz) δ 8.0 (d, 2H), 8.7 (d, 1H), (d, 2H), 7.6 (d, 1H), 4.4 (s, 4H), 2.6 (m, 2H), 2.5 (s, 3H), 1.5 (m, 2H), 0.9 (m, 3H). MS(ESI) (M⁺+1).

### EXAMPLE 12

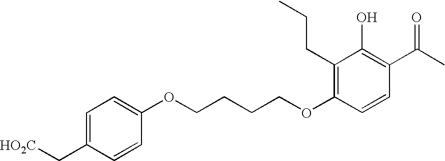

{4-[4-(4-Acetyl-3-hydroxy-2-propyl-phenoxy)-butoxy]-phenyl}-acetic acid was prepared in a similar manner as outlined for 4[4-(4-acetyl-3-hydroxy-2-propyl-phenoxy)-butoxy]-benzoic acid. MS(ESI) 401 (M⁺+1).

### EXAMPLE 13

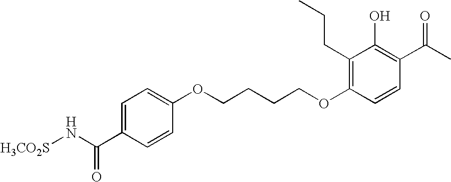

### N-{4-[4-(4-Acetyl-3-hydroxy-2-propyl-phenoxy)-butoxy]-benzoyl}-methanesulfonamide

Freshly distilled oxalyl chloride (0.23 ml, 2.7 mmol) was added to a stirred solution of 4-[4-(4-acetyl-3-hydroxy-2-propyl-phenoxy)-butoxy]-benzoic acid (208 mg, 0.54 mmol) in dichloromethane (13 ml) at rt. Dimethylformamide (0.05 ml) was added dropwise until bubbling ceased and mixture was allowed to stir until no starting material was observed by tlc. Reaction mixture was concentrated in vacuo to give the acid chloride as a brown solid. The acid chloride was dissolved in tetrahydrofuran (5.8 ml) and added to a cooled mixture of sodium hydride (65 mg, 1.6 mmol, methane sulfonamide (128 mg, 1.3 mmol) in tetrahydrofuran (1.0 ml) at 0° C. The mixture was allowed to warm to rt. Addition of sodium hydride in 20 mg portions (twice) over a three hour period was needed to complete the reaction where upon reaction was cooled to 0° C. and quenched with water (1.0 ml). The mixture was washed with brine (2×20 ml) and extracted with ethyl acetate (2×20 ml). Combined organic extracts were dried over Na₂SO₄, filtered and concentrated in vacuo to give a brown oil. The oil was purified by column chromatography on silica gel (eluting first with 30-100% ethyl acetate/hexanes followed by 5-25% methanol/ethyl actetate) to give N-{4-[4-(4-acetyl-3-hydroxy-2-propyl-phenoxy)-butoxy]-benzoyl}-methanesulfonamide. ¹H NMR (CDCl₃, 500 MHz), 12.87 (s, 1H), 7.81 (m, 2H), 7.29 (d, 1H), 7.05 (m, 2H), 6.43 (d, 1H), 4.10 (m, 4H), 3.49 (s, 3H), 2.64 (m, 2H), 2.56 (s, 3H), 2.05 (m, 4H), 1.55 (m, 2H), 0.94 (t, 3H). MS (ESI): 464 (M+H)⁺.

### EXAMPLE 14

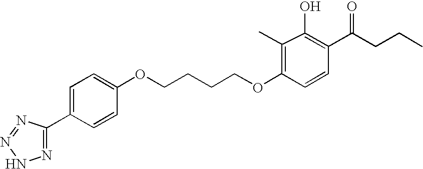

### 1-(2-Hydroxy-3-methyl-4-{4-[4-(2H-tetrazol-5-yl)-phenoxy]-butoxy}-phenyl)-butan-1-one

Butyryl chloride (1.11 g, 1.08 mL, 10.4 mmol) was added to a stirred solution of 2-methylresorcinol (1 g, 8.0 mmol) and aluminum trichloride (1.39 g, 10.4 mmol) in dichloromethane (40 mL) at 0° C. The reaction was allowed to warm to rt, then stirred for 16 hr. It was then quenched by addition of 1N aqueous HCl. The organic layer was separated, dried over MgSO₄ and then concentrated in vacuo to give a residue that was purified via column chromatography on silica gel (eluting 5-60% ethyl acetate/hexanes) to give 824 mg (53%) of 1-(2,4-Dihydroxy-3-methyl-phenyl)-butan-1-one as a white solid.

Potassium carbonate (569 mg, 4.12 mmol) was added to a stirred solution of 1-(2,4-Dihydroxy-3-methyl-phenyl)-butan-1-one (400 mg, 2.06 mmol) and 4-(4-Bromo-butoxy)-benzonitrile (628 mg, 2.47 mmol) in acetone (30 mL) at 45° C. The reaction mixture was stirred for 16 hr, then the acetone was removed in vacuo. The residue was then mixed with dichloromethane (100 mL) and water (100 mL). The organic layer was separated, dried over MgSO₄ and then concentrated in vacuo to give a residue that was purified via column chromatography on silica gel (eluting 5-50% ethyl acetate/hexanes) to give 605 mg (80%) of 4-[4-(4-Butyryl-3-hydroxy-2-methyl-phenoxy)-butoxy]-benzonitrile as a white solid. 4-[4-(4-Butyryl-3-hydroxy-2-methyl-phenoxy)-butoxy]-benzonitrile (400 mg, 1.09 mmol), trimethylsilylazide (251 mg, 0.29 mL, 2.2 mmol) and dibutyltin oxide (41 mg, 0.16 mmol) were dissolved in toluene (15 mL) and heated to reflux for 16 hr. The reaction mixture was then cooled to rt and applied directly to a silica gel column (eluting first with 20% ethyl acetate/hexanes followed by 10% MeOH/dichloromethane) to give 1-(2-Hydroxy-3-methyl-4-{4-[4-(2H-tetrazol-5-yl)-phenoxy]-butoxy}-phenyl)-butan-1-one as a white solid. ¹H NMR (DMSO-d₆, 500 MHz), δ 13.02 (s, 1H), 7.96 (d, 2H), 7.85 (d, 1H), 7.14 (d, 21), 6.66 (d, 1H), 4.18-4.15 (m, 4H), 2.98 (t, 21′), 2.00 (s, 3H), 1.96-1.93 (m, 4H), 1.67-1.63 (m, 2H), 0.95 (t, 3H). MS (ESI): 411 (M+H)⁺.

### EXAMPLE 15

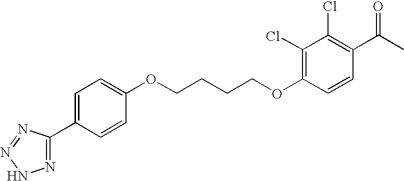

### 1-(2,3-Dichloro-4-{4-[4-(1H-tetrazol-5-yl)phenoxy]-butoxy}-phenyl)ethanone

A mixture of 1-(2,3-dichloro-4-methoxy-phenyl)-ethanone (1.1 g, 5.0 mmol) and pyridine hydrochloride (6.0 g, 51.9 mmol was heated to 180° C. under nitrogen for two hours. After cooling the black residue to rt, water (30 ml) was added and the mixture was extracted with dichloromethane (3×30 ml). The organic layers were dried over Na₂SO₄, filtered and concentrated in vacuo to give a dark pink oil. The oil was purified by column chromatography on silica gel (eluting 1-20% ethyl acetate/hexanes) to afford 1-(2,3-dichloro-4-hydroxy-phenyl)-ethanone as a white solid (297 mg, 30%). Potassium carbonate (138 mg, 1.0 mmol) was added to a stirred solution of 1-(2,3-dichloro-4-hydroxy-phenyl)-ethanone (138 mg, 0.67 mmol) and 4-(4-bromo-butoxy)-benzonitrile (188 mg, 0.74 mmol) in acetone (6.7 ml) at 45° C. The mixture was stirred until disappearance of starting material by tlc where upon the reaction was concentrated in vacuo. The resulting residue was washed with water (2×15 ml) and extracted with dichloromethane (3×20 ml). Organic extracts were dried over Na₂SO₄, filtered and concentrated in vacuo to give a white solid which was purified by column chromatography on silica gel (eluting 0-50% ethyl acetate/hexanes) to afford 4-[4-(4-acetyl-2,3-dichloro-phenoxy)butoxy]-benzonitrile as a white solid (200 mg, 79%). A mixture of 4-[4-(4-acetyl-2,3-dichloro-phenoxy)butoxy]-benzonitrile (188 mg, 0.5 mmol), toluene (7.2 ml), azidotrimethylsilane (0.4 ml, 3.0 mmol) and dibutyl tin oxide (24 mg, 0.09 mmol) was stirred at 110° C. for 16 hr and then cooled to rt. The reaction mixture was purified by column chromatography on silica gel (eluting first with 30-100% ethyl acetate/hexanes followed by 5-20% methanol/ethyl acetate) to give 1-(2,3-dichloro-4-{4-[4-(1H-tetrazol-5-yl)phenoxy]-butoxy}-phenyl)ethanone as a beige solid. ¹H NMR (DMSO-d₆ 500 MHz) 8.27 (d, 2H), 7.77 (d, 1H), 7.28 (d, 1H), 7.16 (d, 2H), 4.27 (m, 2H), 4.17 (m, 2H), 2.56 (s, 3H), 1.96 (m, 4H). MS(ESI): 421 M+.

### EXAMPLE 16

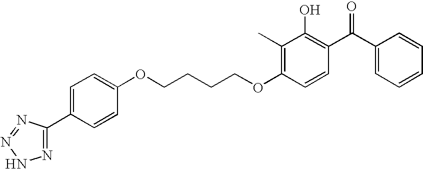

### (2-Hydroxy-3-methyl-4-{4-[4-(2H-tetrazol-5-yl)-phenoxy]-butoxy}-phenyl)-phenyl-methanone

Benzoyl chloride (1.46 g, 1.21 mL, 10.4 mmol) was added to a stirred solution of 2-methylresorcinol (1 g, 8.0 mmol) and aluminum trichloride (1.39 g, 10.4 mmol) in dichloromethane (40 mL) at 0° C. The reaction was allowed to warm to rt, then stirred for 16 hr. It was then quenched by addition of 1N aqueous HCl. The organic layer was separated, dried over MgSO₄ and then concentrated in vacuo to give a residue that was purified via column chromatography on silica gel (eluting 5-60% ethyl acetate/hexanes) to give 902 mg (50%) of (2,4-Dihydroxy-3-methyl-phenyl)-phenyl-methanone as a white solid. Potassium carbonate (752 mg, 5.44 mmol) was added to a stirred solution of (2,4-Dihydroxy-3-methyl-phenyl)-phenyl-methanone (621 mg, 2.72 mmol) and 4-(4-Bromo-butoxy)-benzonitrile (830 mg, 3.26 mmol) in acetone (40 mL) at 45° C. The reaction mixture was stirred for 16 hr, then the acetone was removed in vacuo. The residue was then mixed with dichloromethane (100 mL) and water (100 mL). The organic layer was separated, dried over MgSO₄ and then concentrated in vacuo to give a residue that was purified via column chromatography on silica gel (eluting 1-30% ethyl acetate/hexanes) to give 394 mg (36%) of 4-[4-(4-Benzoyl-3-hydroxy-2-methyl-phenoxy)-butoxy]-benzonitrile as a white solid. 4-[4-(4-Benzoyl-3-hydroxy-2-methyl-phenoxy)-butoxy]-benzonitrile (200 mg, 0.50 mmol), trimethylsilylazide (115 mg, 0.13 mL, 1.0 mmol) and dibutyltin oxide (19 mg, 0.075 mmol) were dissolved in toluene (10 mL) and heated to reflux for 16 hr. The reaction mixture was then cooled to rt and applied directly to a silica gel column (eluting first with 20% ethyl acetate/hexanes followed by 10% MeOH/dichloromethane) to give 81 mg (36%) of (2-Hydroxy-3-methyl-4-{4-[4-(2H-tetrazol-5-yl)-phenoxy]-butoxy}-phenyl)-phenyl-methanone as a white solid. ¹H NMR (DMSO-d₆, 500 MHz), δ 12.53 (s, 1H), 7.95 (d, 2H), 7.67-7.63 (m, 3H), 7.56 (d, 2H), 7.40 (d, 1H), 7.15 (d, 2H), 6.69 (d, 1H), 4.19-4.15 (m, 4H), 2.07 (s, 3H), 1.97-1.95 (m, 4H). MS (ESI): 445 (M+H)⁺.

### EXAMPLE 17

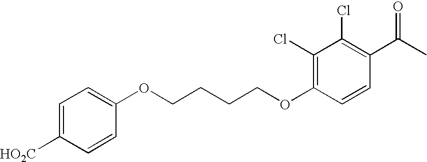

### 4-[4-(4-Acetyl-2,3-dichloro-phenoxy)-butoxy]-benzoic acid

Potassium carbonate (130 mg, 0.95 mmol) was added to a mixture of 1-(2,3-dichloro-4-hydroxy-phenyl)-ethanone (130 mg, 0.63 mmol), 4-(4-bromo-butoxy)-benzoic acid methyl ester (200 mg, 0.70 mmol) in acetone (6.3 ml) at 45° C. The reaction was allowed to stir for 16 hr and then cooled to rt. The mixture was concentrated in vacuo and then washed with water (2×15 ml) and extracted with dichloromethane (3×15 ml). Organic layers were dried over Na₂SO₄, filtered and concentrated in vacuo to give an oil. The crude oil was purified by column chromatography on silica gel (eluting 0-50% ethyl acetate/hexanes) to give 4-[4-(4-acetyl-2,3-dichloro-phenoxy)-butoxy]-benzoic acid methyl ester as a white solid (108 mg, 41%). A mixture of 4-[4-(4-acetyl-2,3-dichloro-phenoxy)-butoxy]-benzoic acid methyl ester (108 mg, 0.26 mmol) in tetrahydrofuran (1.3 ml) and 1.0 N lithium hydroxide aqueous solution (1.3 ml) was stirred at 75° C. for 16 hr and then cooled to rt. Mixture was acidified to pH 1 with 1.0 N HCl aqueous solution and extracted with ethyl acetate (3×20 ml). The organic extracts were dried over Na₂SO₄, filtered and concentrated in vacuo to afford 4-[4-(4-acetyl-2,3-dichloro-phenoxy)-butoxy]-benzoic acid as a yellow solid. ¹H NMR (MSO-d₆ 500 MHz) 12.61 (s, 1H), 7.88 (d, 2H), 7.76 (d, 1H), 7.27 (d, 1H), 7.02 (d, 2H), 4.26 (m, 2H), 4.15 (m, 2H), 2.57 (s, 3H), 1.94 (m, 4H).

### EXAMPLE 18

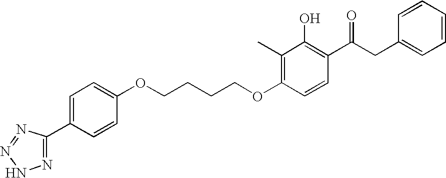

### 1-(2-Hydroxy-3-methyl 4-{4-[4-(2H-tetrazol-5-yl)-phenoxy]-butoxy}-phenyl)-2-phenyl-ethanone

Phenylacetyl chloride (1.61 g, 1.38 mL, 10.4 mmol) was added to a stirred solution of 2-methylresorcinol (1 g, 8.0 mmol) and aluminum trichloride (1.39 g, 10.4 mmol) in dichloromethane (40 mL) at 0° C. The reaction was allowed to warm to rt, then stirred for 16 hr. It was then quenched by addition of 1N aqueous HCl. The organic layer was separated, dried over MgSO₄ and then concentrated in vacuo to give a residue that was purified via column chromatography on silica gel (eluting 1-30% ethyl acetate/hexanes) to give 815 mg (42%) of 1-(2,4-Dihydroxy-3-methyl-phenyl)-2-phenyl-ethanone as a white solid. Potassium carbonate (456 mg, 3.3 mmol) was added to a stirred solution of 1-(2,4-Dihydroxy-3-methyl-phenyl)-2-phenyl-ethanone (400 mg, 1.65 mmol) and 4-(4-Bromo-butoxy)-benzonitrile (505 mg, 1.98 mmol) in acetone (30 mL) at 45° C. The reaction mixture was stirred for 16 hr, then the acetone was removed in vacuo. The residue was then mixed with dichloromethane (100 mL) and water (100 mL). The organic layer was separated, dried over MgSO₄ and then concentrated in vacuo to give a residue that was purified via column chromatography on silica gel (eluting 1-30% ethyl acetate/hexanes) to give 151 mg (23%) of 4-[4-(3-Hydroxy-2-methyl-4-phenylacetyl-phenoxy)-butoxy]-benzonitrile as a white solid. 4-(4-(3-Hydroxy-2-methyl-4-phenylacetyl-phenoxy)-butoxy]-benzonitrile (75 mg, 0.18 mmol), trimethylsilylazide (42 mg, 0.05 mL, 0.36 mmol) and dibutyltin oxide (6 mg, 0.027 mmol) were dissolved in toluene (5 mL) and heated to reflux for 16 hr. The reaction mixture was then cooled to rt and applied directly to a silica gel column (eluting first with 20% ethyl acetate/hexanes followed by 10% MeO/dichloro-methane) to give 48 mg (58%) of 1-(2-Hydroxy-3-methyl-4-{4-[4-(2H-tetrazol-5-yl)-phenoxy]-butoxy}-phenyl)-2-phenyl-ethanone as a white solid. ¹H NMR (DMSO-d₆, 500 MHz), δ 12.76 (s, 1H), 8.01 (d, 1H), 7.95 (d, 2H), 7.34-7.24 (m, 5H), 7.12 (d, 2H), 6.69 (d, 1H), 4.36 (s, 2H), 4.19-4.13 (m, 4H), 2.00 (s, 3H), 1.94-1.91 (m, 4H). MS (ESI): 459 (M+H)⁺.

### EXAMPLE 19

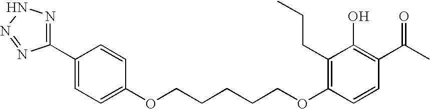

### 1-[2-Hydroxy-3-propyl-4-({5-[4-(1H-tetraazol-5-yl)phenoxy]pentyl}oxy)phenyl]-ethanone

To stirred a solution of 4-[(5-bromopentyl)oxy]benzonitrile (2.5 g, 0.9 mmol) in acetone (50 ml) was added K₂CO₃ (2.6 g, 1.7 mmol) and 2′4′-dihydroxy-3′-propylacetophenone (1.9 g, 1.0 mmol). The reaction mixture was allowed to stir at 60° C. for 18 hours, cooled to ambient temperature and filtered. The filtrate was concentrated in vacuo and the crude product was purified by flash chromatography on silica gel eluting with hexanes:EtOAc (4:1) to afford 4-{[5-(4-acetyl-3-hydroxy-2-propylphenoxy)pentyl]oxy}benzonitrile (1.1 g, 50%) as white solid. MS (ESI) 382 (M⁺+1). To a degassed solution of 4-{[5-(4-acetyl-3-hydroxy-2-propylphenoxy)-pentyl]oxy}benzonitrile (300 mg, 0.79 mmol) in toluene (5 ml) was added azidotrimethylsilane (360 mg, 3.1 mmol) and di-n-butyltin oxide (30 mg, 0.1 mmol). The reaction was stirred at 110° C. for 20 hours and cooled to ambient temperature. The mixture was purified using preparative-TLC plate, to afford desired 1-[2-hydroxy-3-propyl-4-({5-[4-(1H-tetraazol-5-yl)phenoxy]pentyl}oxy)phenyl]-ethanone. ¹H NMR (CD₃OD, 500 MH) 68.0 (d, 2H), 7.8 (d, 1H), 7.0 (d, 2H), 6.6 (d, 2H), 4.2 (m, 4H), 2.6 (m, 2H), 2.5 (s, 3H), 2.0 (m, 4H), 1.8 (m, 2H), 1.4 (m, 2H), 0.8 (m, 3H). MS (ESI) 425(M⁺+1)

### EXAMPLE 20

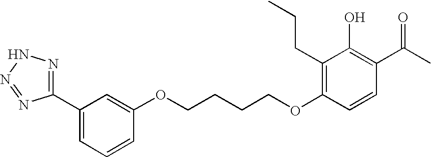

### 1-(2-Hydroxy-3-propyl-4-{4-[3-(1H-tetraazol-5-yl)phenoxy]butoxy}phenyl)ethanone

To stirred a solution of 3-(4-bromobutoxy)benzonitrile (2.1 g, 0.84 mmol) in acetone (50 ml) was added K₂CO₃ (2.3 g, 1.7 mmol) and 2′4′-dihydroxy-3′-propylacetophenone (1.7 g, 0.89 mmol). The reaction mixture was allowed to stir at 60° C. for 18 hours, cooled to ambient temperature and filtered. The filtrate was concentrated in vacuo and the crude product was purified by flash chromatography on silica gel eluting with hexanes:EtOAc (4:1) to afford 3-[4-(4-acetyl-3-hydroxy-2-propylphenoxy)butoxy]benzonitrile (1.7 g, 54%) as white solid. MS (ESI) 368(M⁺+1).

To a degassed solution of 3-[4-(4-acetyl-3-hydroxy-2-propylphenoxy)butoxy]benzonitrile (200 mg, 0.54 mmol) in toluene (3 ml) was added azidotrimethylsilane (250 mg, 2.1 mmol) and di-n-butyltin oxide (20 mg, O. 1 mmol). The reaction mixture was stirred at 110° C. for 20 hours and cooled to ambient temperature. The mixture was purified using preparative TLC plate to afford 1-(2-hydroxy-3-propyl-4-{4-[3-(1H-tetraazol-5-yl)phenoxy]butoxy}phenyl)ethanone. ¹H NMR (CD₃OD, 500 MHz) δ 7.7 (d, 1H), 7.5 (m, 2H), 7.3 (m, 1H), 6.9 (d, 1H), 6.6 (d, 2H), 4.2 (m, 4H), 2.6 (m, 2H), 2.5 (s, 3H), 2.1 (m, 4H), 1.5 (m, 2H), 0.9 (m, 3H). MS (ESI) 411 (M⁺+1)

### EXAMPLE 21

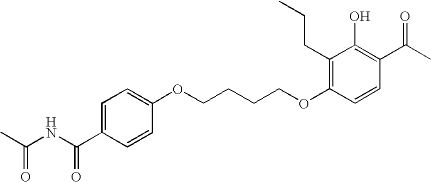

### N-Acetyl-4-[4-(4-acetyl-3-hydroxy-2-propyl-phenoxy)-butoxy]-benzamide

Freshly distilled oxalyl chloride (0.2 ml, 2.3 mmol) was added to a stirred solution of 4-[4-(4-acetyl-3-hydroxy-2-propyl-phenoxy)-butoxy]-benzoic acid (194 mg, 0.5 mmol) in dichloromethane (13 ml) at rt. Dimethylformamide (0.1 ml) was added dropwise until bubbling ceased and mixture was allowed to stir until no starting material was observed by tlc. Reaction mixture was concentrated in vacuo to give the acid chloride as a brown solid. The acid chloride was dissolved in tetrahydrofuran (5.0 ml) and added to a cooled mixture of sodium hydride (68 mg, 1.7 mmol) and acetamide (77 mg, 1.3 mmol) in tetrahydrofuran (1.0 ml) at 0° C. Reaction was allowed to warm to rt overnight and then washed with water (70 ml) and extracted with ethyl acetate (60 ml). Organic layer was dried over Na₂SO₄, filtered and concentrated in vacuo to give a brown residue. The crude residue was purified by column chromatography on silica gel (eluting 30-50% ethyl acetate/hexanes) to give N-acetyl-4-[4-(4-acetyl-3-hydroxy-2-propyl-phenoxy)-butoxy]-benzamide as a white solid. ¹H NMR (DMSO-d₆, 500 MHz), 12.85 (s, 1H), 10.86 (s, 1H), 7.94 (d, 2H), 7.82 (d, 1H), 7.05 (d, 2H), 6.67 (d, 1H), 4.17 (m, 4H), 2.59 (s, 3H), 2.54 (m, 2H), 2.35 (s, 3H), 1.93 (m, 4H), 1.47 (m, 2H), 0.88 (t, 3H). MS (ESI): 428 (M+H)⁺.

### EXAMPLE 22

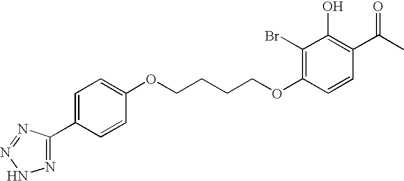

### 1-(3-Bromo-2-hydroxy-4-{4-[4-(1H-tetraazol-5-yl)phenoxy]butoxy}phenyl)ethanone

Bromine (10.5 g, 66 mmol) was added dropwise to a solution of 2′4′-dihydroxyacetophenone (10 g, 66 mmol) in EtOH (250 ml) at −70° C. The reaction mixture was then warmed to ambient temperature, stirred for 2 hours and concentrated in vacuo. The crude material was purified by flash chromatography on silica gel eluting with hexanes:EtOAc (3:1) to afford 1-(3-bromo-2,4-dihydroxyphenyl)-ethanone (5.8 g, 35%). MS (ESI) 232(M⁺+1). To a stirred solution of 4-(4-bromobutoxy)benzonitrile (560 mg, 2.2 mmol) in acetone (50 ml) was added K₂CO₃ (607 mg, 4.4 mmol) and 1-(3-bromo-2,4-dihydroxyphenyl)ethanone (511 g, 2.2 mmol). The mixture then was allowed to stir at 40° C. for 20 hours, cooled to ambient temperature, and filtered. The filtrate was concentrated in vacuo and the crude product was purified by flash chromatography on silica gel eluting with hexanes:EtOAc (3:1) to afford 4-[4-(4-acetyl-2-bromo-3-hydroxyphenoxy)-butoxy]benzonitrile (230 mg, 26%) as white solid. MS (ESI) 403(M⁺+1). To a degassed solution of afford 4-[4-(4-acetyl-2-bromo-3-hydroxyphenoxy)butoxy]benzonitrile (44 mg, 0.11 mmol) in toluene (2 ml) was added azidotrimethylsilane (50 mg, 0.44 mmol), di-n-butyltin oxide (20 mg, 0.08 mmol). The reaction was stirred at 110° C. for 20 hours and cooled to ambient temperature. The mixture was purified using preparative TLC plate to afford desired 1-(3-bromo-2-hydroxy-4-{4-[4-(1H-tetraazol-5-yl)phenoxy]butoxy}phenyl)ethanone. ¹H NMR (CD₃OD, 500 MHz) δ 8.0 (d, 2H), 7.8 (m, 1H), 7.0 (d, 2H), 6.6 (d, 2H), 4.2 (m, 4H), 2.6 (s, 3H), 2.0 (m, 4H). MS (ESI) 448(M⁺+1).

### EXAMPLE 23

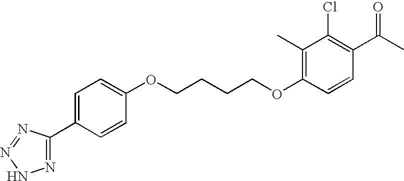

### 1-(2-Chloro-3-methyl-4-{4-[4-(2H-tetrazol-5-yl)-phenoxy]-butoxy}-phenyl)-ethanone

Prepared in a similar fashion as outlined in example 15 using 1-(2-Chloro-4-hydroxy-3-methyl-phenyl)-ethanone. ¹H NMR (DMSO-d₆, 500 mHz) δ 7.97 (d, 2H), 7.59 (d, H), 7.14 (d, 2H), 7.06 (d, 1H), 4.16 (dd, 4H), 2.54 (s, 3H), 2.23 (s, 3H), 1.99 (dd, 4H). ESI: 401 (M+H)⁺

### EXAMPLE 24

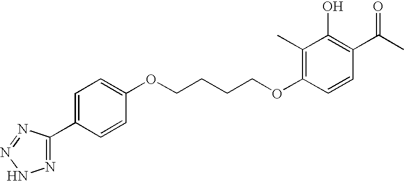

### 1-(2-Hydroxy-3-methyl-4-{4-[4-(2H-tetrazol-5-yl)-phenoxy]-butoxy}-phenyl)-propan-1-one

Prepared in a similar fashion as outlined in example 3 using propanoic acid chloride. ¹H NMR (DMSO-d₆, 500 MHz) δ 12.70 (s, 1H), 7.95 (d, 2H), 7.83 (d, 1H), 7.13 (d, 2H), 6.66 (d, 1H), 4.18-4.13 (m, 4H), 2.04 (q, 2H), 1.99 (s, 3H), 1.97-1.92 (m, 4H), 1.10 (t, 3H). MS (ES) 397(M⁺+1).

### EXAMPLE 25

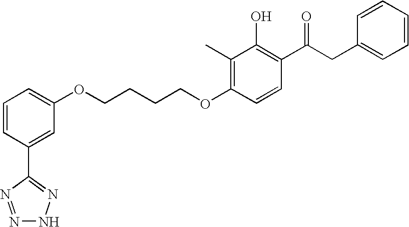

### 1-(2-Hydroxy-3-methyl-4-{4-[3-(2H-tetrazol-5-yl)-phenoxy]-butoxy}-phenyl)-2-phenylethanone

Prepared in a similar fashion as outlined in example 18 using 3-(4-bromobutoxy)benzonitrile. ¹H NMR (DMSO-d₆, 500 MHz) δ 12.76 (s, 1H), 8.02 (d, 1H), 7.60-7.57 (m, 2H), 7.44 (t, 1H), 7.33-7.25 (m, 5H), 7.07-7.04 (m, 1H), 6.69 (d, 1H), 4.36 (s, 2H), 4.20-4.13 (m, 4H), 1.99 (s, 3H), 1.96-1.93 (m, 4H). MS (ESI) 459 (M⁺+1).

### EXAMPLE 26

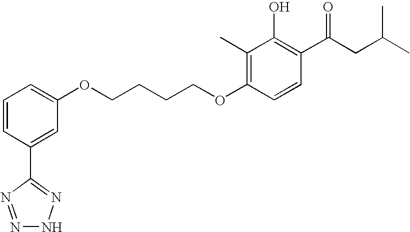

### 1-(2-Hydroxy-3-methyl-4-{4-[3-(2H-tetrazol-5-yl)-phenoxy]-butoxy}-phenyl)-3-methyl-butan-1-one

Prepared in a similar fashion as outlined in example 6 using 3-(4-bromobutoxy)benzonitrile. ¹H NMR (DMSO-d₆, 500 MHz) δ 13.02 (s, 1H), 7.84 (d, 1H), 7.62-7.58 (m, 2H), 7.49 (t, 1H), 7.15-7.12 (m, 1H), 6.66 (d, 1H), 4.20-4.14 (m, 4H), 2.85 (d, 2H), 2.17-2.12 (m, 1H), 1.99 (s, 3H), 1.97-1.92 (m, 4H), 0.94 (d, 6H). MS (ESI) 425 (M⁺+1).

### EXAMPLE 27

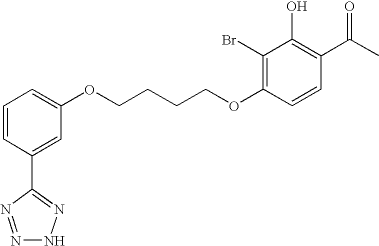

### 1-(3-Bromo-2-hydroxy-4-{4-[3-(2H-tetrazol-5-yl)-phenoxy]-butoxy}-phenyl)-ethanone

Prepared in a similar fashion as outlined in example 22 using 3-(4-bromobutoxy)benzonitrile. ¹H NMR δ 7.85 (d, 1H), 7.65 (m, 2H), 7.35 (t, 1H) 6.95 (m, 1H), 6.60 (d, 1H), 4.40 (m, 4H), 2.60 (s, 3H), 2.1 (m, 4H). MS 448.8(M+1)

### EXAMPLE 28

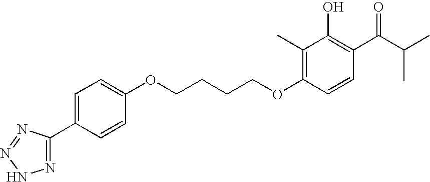

### 1-(2-Hydroxy-3-methyl-4-{4-[4-(2H-tetrazol-5-yl)-phenoxy]-butoxy}-phenyl)-2-methyl-propan-1-one

Prepared in a similar fashion as outlined in example 3 using isobutyryl chloride. ¹H NMR (DMSO-d₆, 500 MHz) δ 13.05 (s, 1H), 7.95 (d, 2H), 7.89 (d, 1H), 7.14 (d, 2H), 6.67 (d, 1H), 4.19-4.14 (m, 4H), 3.72-3.68 (m, 1H), 2.00 (s, 3H), 1.97-1.92 (m, 4H), 1.13 (d, 6H). MS (ESI) 411 (M⁺+1).

### EXAMPLE 29

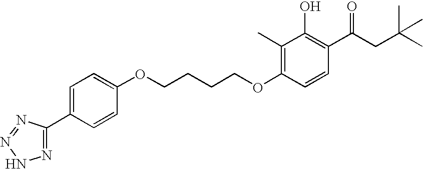

### 1-(2-Hydroxy-3-methyl-4-{4-[4-(2H-tetrazol-5-yl)-phenoxy]-butoxy}-phenyl)-3,3-dimethyl-butan-1-one

Prepared in a similar fashion as outlined in example 3 using tert-butylacetyl chloride. ¹H NMR (DMSO-d₆, 500 MHz) δ 13.24 (s, 1H), 7.95 (d, 2H), 7.90 (d, 1H), 7.12 (d, 2H), 6.64 (d, 1H), 4.19-4.14 (m, 4H), 2.86 (s, 2H), 2.00 (s, 31), 1.97-1.92 (m, 4H), 1.01 (s, 9H). MS (ESI) 439 (M⁺+1).

### EXAMPLE 30

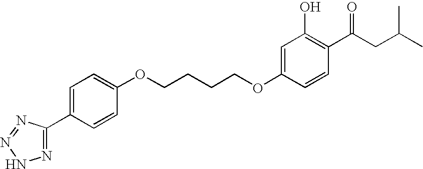

### 1-(2-Hydroxy-4-{4-[4-(2H-tetrazol-5-yl)-phenoxy]-butoxy}-phenyl)-3-methyl-butan-1-one

Prepared in a similar fashion as outlined in example 3 using resorcinol. ¹H NMR (DMSO-d₆, 500 MHz) δ 12.80 (s, 1H), 7.96 (d, 2H), 7.87 (d, 1H), 7.15 (d, 2H), 6.54-6.48 (m, 2H), 4.15-4.12 (m, 4H), 2.84 (d, 2H), 2.16-2.11 (m, 1H), 1.91-1.88 (m, 4H), 0.94 (d, 6H). MS (ESI) 411 (M⁺+1).

### EXAMPLE 31

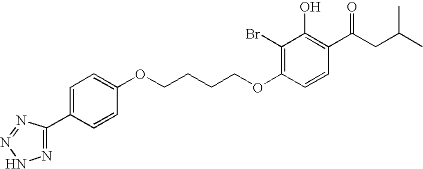

### 1-(3-Bromo-2-hydroxy-4-{4-[4-(2H-tetrazol-5-yl)-phenoxy]-butoxy}-phenyl)-3-methyl-butan-1-one

Prepared in a similar fashion as outlined in example 22 using 1-(2,4-Dihydroxy-phenyl)-3-methyl-butan-1-one. ¹H NMR (DMSO-d₆, 500 MHz) δ 13.48 (s, 1H), 8.04 (d, 1H), 7.93 (d, 2H), 7.07 (d, 2H), 6.79 (d, 1H), 4.31-4.28 (m, 2H), 4.16-4.12 (m, 2H), 2.92 (d, 2H), 2.19-2.13 (m, 1H), 1.97-1.91 (m, 4H), 0.96 (d, 6H). MS (ESI) 490 (M⁺+1).

### EXAMPLE 32

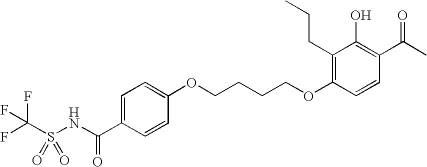

### N-{4-[4-(4-Acetyl-3-hydroxy-2-propyl-phenoxy)-butoxy]-benzoyl}-C,C,C-trifluoro-methanesulfonamide

Prepared in a similar fashion as outlined in example 13 using trifluoromethanesulfonamide. ¹H NMR (DMSO-d₆, 500 MHz) δ 12.89 (s, 1H), 7.85 (d, 2H), 7.81 (d, 1H), 6.90 (d, 2H), 6.66 (d, 1H), 4.15 (dd, 2H), 4.08 (dd, 2H), 2.57 (s, 3H), 2.53 (m, 2H). 2.12 (s, 1H), 1.90 (m, 4H), 1.46 (q, 2H), 0.85 (t, 3H). ESI: 518 (M+H)⁺

### EXAMPLE 33

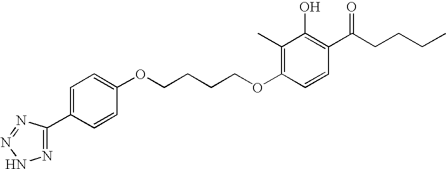

### 1-(2-Hydroxy-3-methyl-4-{4-[4-(2H-tetrazol-5-yl)-phenoxy]-butoxy}-phenyl)-pentan-1-one

Prepared in a similar fashion as outlined in example 3 using pentanoic acid chloride. ¹H NMR (DMSO-d₆, 500 MHz) δ 12.94 (s, 1H), 7.95 (d, 2H), 7.84 (d, 1H), 7.11 (d, 2H), 6.65 (d, 1H), 4.19-4.13 (m, 4H), 3.00 (t, 2H), 2.01 (s, 3H), 1.97-1.93 (m, 4H), 1.63-1.58 (m, 2H), 1.37-1.31 (m, 2H), 0.88 (t, 3H). MS (ESI) 424 (M⁺+1).

### EXAMPLE 34

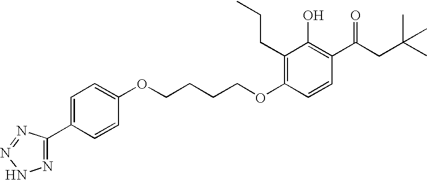

### 1-(2-Hydroxy-3-propyl-4-{4-[4-(2H-tetrazol-5-yl)-phenoxy]-butoxy}-phenyl)-3,3-dimethyl-butan-1-one

Prepared in a similar fashion as outlined in example 3 using 1-(2,4-Dihydroxy-3-propyl-phenyl)-3,3-dimethyl-butan-1-one. ¹H NMR (DMSO-d₆, 500 MH) δ 13.25 (s, 1H), 7.95 (d, 21), 7.92 (d, 1H), 7.11 (d, 2H), 6.64 (d, 1H), 4.18-4.13 (m, 4H), 2.86 (s, 2H), 2.54 (t, 2H), 1.94-1.91 (m, 4H), 1.02 (s, 9H), 0.87 (t, 3H). MS (ESI) 467 (M⁺+1).

### EXAMPLE 35

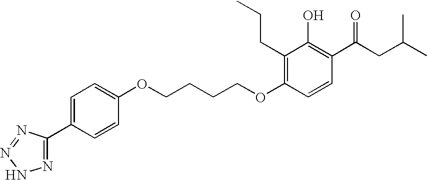

### 1-(2-Hydroxy-3-propyl-4-{4-[4-(2H-tetrazol-5-yl)-phenoxy]-butoxy}-phenyl)-3-methyl-butan-1-one

Prepared in a similar fashion as outlined in example 3 using 1-(2,4-Dihydroxy-3-propyl-phenyl)-3-methyl-butan-1-one. ¹H NMR (DMSO-d₆, 500 MHz) δ 13.03 (s, 1H), 7.97 (d, 2H), 7.85 (d, 1H), 7.15 (d, 2H), 6.65 (d, 1H), 4.17-4.14 (m, 4H), 2.85 (d, 2H), 2.51 (t, 2H), 2.17-2.12 (m, 1H), 1.94-1.92 (m, 4H), 1.48-1.43 (m, 2H), 0.94 (d, 6H), 0.86 (t, 3H). MS (ESI) 453 (M⁺+1).

### EXAMPLE 36

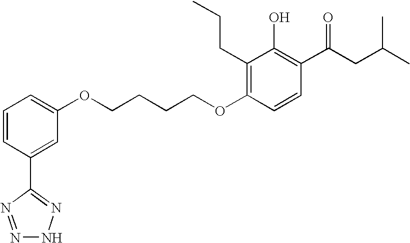

### 1-(2-Hydroxy-3-propyl-4-{4-[3-(2H-tetrazol-5-yl)-phenoxy]-butoxy}-phenyl)-3-methyl-butan-1-one

Prepared in a similar fashion as outlined in example 35 using 3-(4-bromobutoxy)benzonitrile. ¹H NMR (DMSO-d₆, 500 MHz) δ 13.01 (s, 1H), 7.85 (d, 1H), 7.62-7.58 (m, 2H), 7.51-7.48 (m, 1H), 7.15 (dd, 1H), 6.67 (d, 1H), 4.17-4.16 (m. 4H), 2,85 (d, 2H), 2.51 (m, 2H), 2.15 (m, 1H), 1.99-1.94 (m, 4H), 1.46 (q, 2H), 0.95 (s, 311), 0.94 (s, 3H), 0.85 (t, 3H). ESI: 453 (M+H)⁺

### EXAMPLE 37

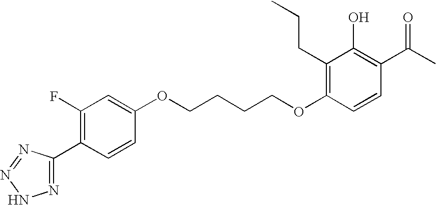

### 1-(4-{4-[3-Fluoro-4-(2H-tetrazol-5-yl)-phenoxy]-butoxy}-2-hydroxy-3-propyl-phenyl)-ethanone

Prepared in a similar fashion as outlined in example 5 using 2-fluoro-4-(4-bromobutoxy)benzonitrile. ¹H NMR (CD₃OD, 500 MHz) δ 7.70 (t, 1H), 7.60 (d, 1H), 6.65 (m, 2H) 6.50 (d, 1H), 4.00 (d, 4H), 2.60 (m, 2H) 2.50 (s, 3H), 1.9 (m, 4H), 1.50 (m, 2H), 0.80 (m, 3H). MS 429.1(M+1)

### EXAMPLE 38

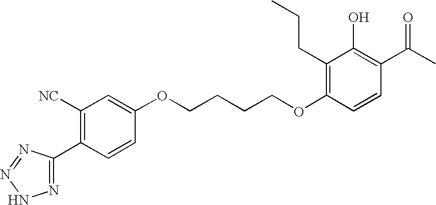

### 5-[4-(4-Acetyl-3-hydroxy-2-propyl-phenoxy)-butoxy]-2-(2H-tetrazol-5-yl)-benzonitrile

Prepared in a similar fashion as outlined in example 5 using 2-cyano-4-(4-bromobutoxy)benzonitrile. ¹H NMR (CD₃OD, 500 MHz) δ 7.65 (t, 2H), 7.50 (d, 1H), 7.10 (d, 1H), 6.65 (m, 1H), 4.00 (d, 4H), 2.60 (m, 2H) 2.50 (s, 3H), 1.9 (m, 4H), 1.50 (m, 2H), 0.80 (m, 3H). MS 436.0(M+1)

### EXAMPLE 39

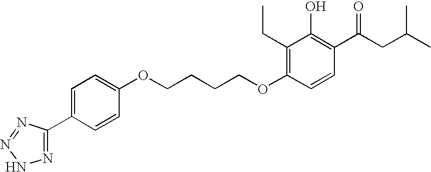

### 1-(3-Ethyl-2-hydroxy-4-{4-[4-(2H-tetrazol-5-yl)-phenoxy]-butoxy}-phenyl)-3-methyl-butan-1-one

Prepared in a similar fashion as outlined in example 3 using 1-(3-Ethyl-2,4-dihydroxy-phenyl)-3-methyl-butan-1-one. ¹H NMR (DMSO-d₆, 500 MHz) δ 13.03 (s, 1H), 7.97 (d, 2H), 7.82 (d, 1H), 7.09 (d, 2H), 6.65 (d, 1H), 4.18-4.04 (m, 4H), 2.86 (d, 2H), 2.56 (q, 2H), 2.18-2.12 (m, 1H), 1.93-1.91 (m, 4H), 1.02 (t, 3H), 0.95 (d, 6H). MS (ESI) 439 (M⁺+1).

### EXAMPLE 40

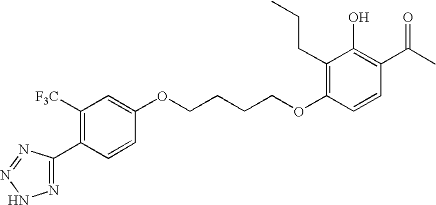

### 1-(2-Hydroxy-3-propyl-4-{4-[4-(2H-tetrazol-5-yl)-3-trifluoromethyl-phenoxy]-butoxy}-phenyl)-ethanone

Prepared in a similar fashion as outlined in example 5 using 2-trifluoromethyl-4-(4-bromobutoxy)benzonitrile. ¹H NMR (CD₃OD, 500 MHz) δ 7.80 (d, 1H), 7.60 (d, 1H), 7.40 (s, H), 7.30 (d, 1H) 6.60 (d, 1H), 4.00 (d, 4H), 2.70 (m, 2H), 2.60 (s, 3H), 2.10 (m, 4H), 1.55 (m, 2H), 0.0 (m, 3H). MS 479(M+1)

### EXAMPLE 41

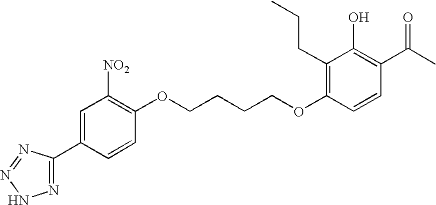

### 1-(2-Hydroxy-3-propyl-4-{4-[4-(2H-tetrazol-5-yl)-2-nitro-phenoxy]-butoxy}-phenyl)-ethanone

Prepared in a similar fashion as outlined in example 5 using 3-nitro-4-(4-bromobutoxy)benzonitrile. ¹H NMR (CD₃OD, 500 MHz) δ 8.50 (s, 1H), 8.25 (d, 1H), 7.55 (d, 1H), 7.40 (d, 1H) 6.60 (d, 1H), 4.30 (m, 2H), 4.10 (m, 2H), 2.70 (t, 2H), 2.60 (s, 3H), 2.10 (m, 4H), 1.50 (m, 2H), 0.90 (m, 3H). MS 456(M+1).

### EXAMPLE 42

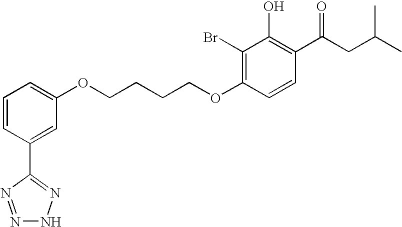

### 1-(3-Bromo-2-hydroxy-4-{4-[3-(2H-tetrazol-5-yl)-phenoxy]-butoxy}-phenyl)-3-methyl-butan-1-one

Prepared in a similar fashion as outlined in example 31 using 3-(4-bromobutoxy)benzonitrile. ¹H NMR (DMSO-d₆, 500 MHz) δ 13.47 (s, 1H), 8.04 (d, 1H), 7.62-7.59 (m, 2H), 7.48 (t, 1H), 7.12-7.10 (m, 1H), 6.79 (d, 1H), 4.31-4.29 (m, 2H), 4.19-4.17 (m, 2H), 2.91 (d, 2H), 2.18-2.13 (m, 1H), 1.99-1.92 (m, 4H), 0.88 (d, 6H). MS (ESI) 489 (M⁺+1).

### EXAMPLE 43

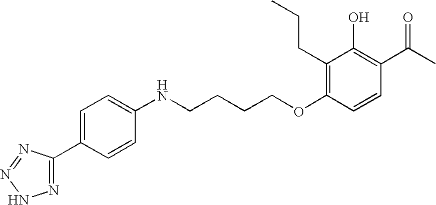

### 1-(2-Hydroxy-3-propyl-4-{4-[4-(2H-tetrazol-5-yl)-phenylamino]-butoxy}-phenyl)-ethanone hydrochloride

To a mixture of 4-aminobenzonitrile (154 mg, 1.3 mmol), 1-[4-(4-bromobutoxy)-2-hydroxy-3-propyl phenyl]ethanone (214 mg, 0.6 mmol) and tetrahydrofuran (6.0 ml) under nitrogen atmosphere was added 0.5 M potassium bis(trimethylsilyl)amide in toluene (2.5 ml, 1.3 mmol) at 0° C. The mixture was allowed to warm to room temperature. After one hour, no starting material was observed by tlc. Mixture was quenched by addition of water and product extracted with ethyl acetate (2×30 ml). Organic extracts were dried (Na₂SO₄), filtered and concentrated in vacuo. The resulting yellow oil was purified by flash chromatography on silica gel (5-60% ethyl acetate/hexane) to give 4-{[4-(4-acetyl-3-hydroxy-2-propylphenoxy)butyl]amino}benzonitrile as a colorless oil (99 mg, 20%). 4-([4-(4-Acetyl-3-hydroxy-2-propylphenoxy)butyl]amino)benzonitrile (99 mg, 0.27 mmol), toluene (3.8 ml), dibutyltin oxide (15 mg, 0.06 mmol) and azidotrimethylsilane (0.22 ml., 1.66 mmol) was heated to 110° C. under nitrogen overnight. Additional heating for three hours and excess azidotrimethyl silane (0.22 ml, 1.66 mol) was needed to complete reaction. Mixture was cooled to room temperature and purified by flash chromatography on silica gel (50-100% ethyl acetate/hexane, followed by 5-25% methanol/ethyl acetate) to afford 1-(2-hydroxy-3-propyl-4-(4-[4-(2H-tetrazol-5-yl)-phenylamino]-butoxy)-phenyl)-ethanone as a yellow solid (57 mg). To a mixture of 1-(2-hydroxy-3-propyl-4-{4-[4-(2H-tetrazol-5-yl)-phenylamino]-butoxy}-phenyl)-ethanone in dichloromethane (2.0 ml) was added 1.0 N HCl in diethyl ether (0.6 ml). Mixture was stirred for a few minutes and concentrated to give 1-(2-hydroxy-3-propyl-4-{4-[4-(2H-tetrazol-5-yl)-phenylamino]-butoxy}-phenyl)-ethanone hydrochloride as a yellow solid. ¹H NMR (DMSO-d₆, 500 MHz) δ 13.03 (s, 1H), 7.81-7.76 (m, 3H), 6.75 (d, 2H), 6.65 (d, 1H), 5.76 (s, 1H), 4.15 (dd, 2H), 3.16 (dd, 2H), 2.58 (s, 3H), 2.53 (m, 2H), 1.83 (m, 2H), 1.74 (m, 2H), 1.49-1.43 (m, 2H), 0.88 (t, 3H). ESI: 410 (M+H)+

### EXAMPLE 44

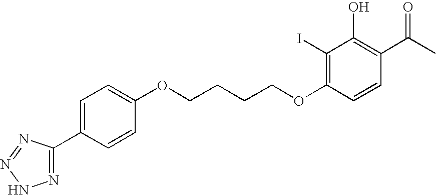

### 1-(2-Hydroxy-3-iodo-4-{4-[4-(2H-tetrazol-5-yl)-phenoxy]-butoxy}-phenyl)-ethanone

Prepared in a similar fashion as outlined in example 22 using iodine. ¹H NMR (DMSO-d₆, 500 MHz) δ 13.52 (s, 1H), 7.99 (d, 1H), 7.95 (d, 2H), 7.13 (d, 2H), 6.71 (d, 1H), 4.27 (t, 2H), 4.16 (t, 2H), 2.63 (s, 3H), 2.00-1.92 (m, 4H). MS (ESI) 494 (M⁺+1).

### EXAMPLE 45

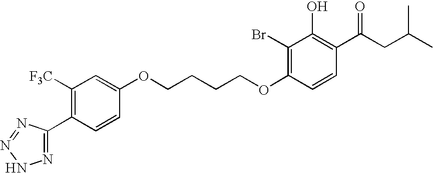

### 1-(3-Bromo-2-hydroxy-4-{4-[4-(2H-tetrazol-5-yl)-3-trifluoromethyl-phenoxy]-butoxy}-phenyl)-3-methyl-butan-1-one

Prepared in a similar fashion as outlined in example 31 using 3-trifluoromethyl-4-(4-bromobutoxy)benzonitrile. ¹H NMR (DMSO-d₆, 500 MHz) δ 13.49 (s, 1H), 8.06 (d, 1H), 7.74 (d, 1H), 7.46-7.44 (m, 2H), 6.81 (d, 1H), 4.32-4.28 (m, 4H), 2.94 (d, 2H), 2.20-2.15 (m, 1H), 2.01-1.99 (m, 4H), 0.97 (d, 6H). MS (ESI) 557 (M⁺+1).

### EXAMPLE 46

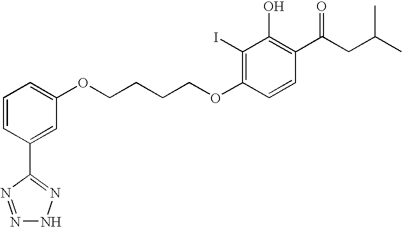

### 1-(2-Hydroxy-3-iodo-4-{4-([3-(2H-tetrazol-5-yl)-phenoxy]-butoxy}-phenyl)-3-methyl-butan-1-one

Prepared in a similar fashion as outlined in example 44 using 3-(4-bromobutoxy)benzonitrile. ¹H NMR (DMSO-d₆, 500 MHz) δ 13.68 (s, 1H), 8.03 (d, 1H), 7.62-7.69 (m, 2H), 7.48 (t, 1H), 7.14-7.12 (m, 1H), 6.69 (d, 1H), 4.27 (t, 2H), 4.18 (t, 2H), 2.90 (d, 2H), 2.17-2.14 (m, 1H), 2.02-1.95 (m, 4H), 0.95 (d, 6H). MS (ESI) 536 (M⁺+1).

### EXAMPLE 47

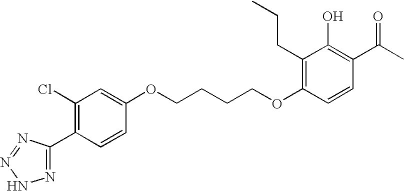

### 1-(4-{4-[3-Chloro-4-(2H-tetrazol-5-yl)-phenoxy]-butoxy}-2-hydroxy-3-propyl-phenyl)-ethanone

Prepared in a similar fashion as outlined in example 5 using 2-chloro-4-(4-bromobutoxy)benzonitrile. ¹H NMR (DMSO-d₆, 500 MHz) δ 12.84, (s, 1H), 7.83 (d, 1H), 7.56 (d, 1H), 7.28 (dd, 1H), 7.13 (dd, 1H), 6.68 (d, 1H), 4.19-4.17 (m, 4H), 2.58 (s, 3H), 2.55 (m, 2H), 1.93 (m, 4H), 1.47 (q, 2H), 0.86 (t, 3H).

ESI: 445 (M+H)+

### EXAMPLE 48

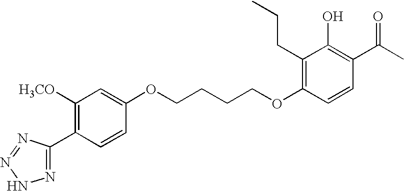

### 1-(2-Hydroxy-4-{4-[3-methoxy-4-(2H-tetrazol-5-yl)-phenoxy]-butoxy}-3-propyl-phenyl)-ethanone

Prepared in a similar fashion as outlined in example 5 using 2-methoxy-4-(4-bromobutoxy)benzonitrile. ¹H NMR (DMSO-d₆, 500 MHz) δ 12.84 (s, 1H), 7.81 (d, 1H), 7.59-7.56 (m, 2H), 7.13 (d, 1H), 6.67 (d, 1H), 4-18-4-12 (m, 4H), 3.84 (s, 3H), 2.58 (s, 3H), 2.55 (m, H), 1.93 (m, 4H), 1.48-1.44 (m, 2H), 0.85 (t, 3H).

ESI: 441 (M+H)⁺

### EXAMPLE 49

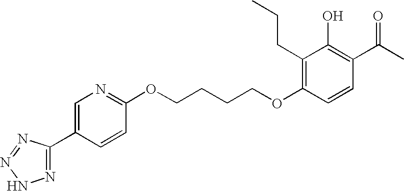

### 1-(2-Hydroxy-3-propyl-4-{4-[5-(2H-tetrazol-5-yl)-pyridin-2-yloxy]-butoxy}-phenyl)-ethanone

A mixture of 6-chloronicotinonitrile (390 mg, 2.8 mmol), dimethylformamide (28 ml). 1,4-butanediol (1.0 ml, 11.3 mmol) and potassium carbonate (506 mg, 3.7 mmol) was heated to 140° C. for ten minutes. The reaction was cooled, washed with brine (100 ml) and extracted with ethyl acetate (3×40 ml). The organic extracts were dried (Na₂SO₄), filtered and concentrated in vacuo. Purification of the resulting oil using flash chromatography on silica gel (0-100% ethyl acetate/hexanes) afforded 6-(4-hydroxybutoxy) nicotinonitrile as a waxy white solid (240 mg, 44%). A mixture of 6-(4-hydroxybutoxy) nicotinonitrile (239 mg, 1.2 mmol), tetrahydrofuran (4.9 ml) and triphenylphosphine (487 mg, 1.8 mmol) was stirred at −5° C. under nitrogen atmosphere. Diethylazodicarboxylate (0.29 ml, 1.8 mmol) was added and mixture was allowed to warm to room temperature. 2′,4′-Dihydroxy-3′-propylacetophenone (458 mg, 2.4 mmol) was then added. Mixture stirred for 16 hours at room temperature and then concentrated in vacuo to give a brown oil which was purified by flash chromatography on silica gel (0-60% ethyl acetate/hexanes) to give a 1:1 mixture of 2′,4′-dihydroxy-3′-propylaceto-phenone and 6-[4-(4-acetyl-3-hydroxy-2-propylphenoxy)butoxy]nicotinonitrile (630 mg.) A mixture of the crude product (630 mg, 0.91 mmol), toluene (13 ml), dibutyltin oxide (34 mg, 0.14 mmol) and azidotrimethylsilane (0.72 ml, 5.5 mmol) was heated overnight at 110° C. Purification of cooled reaction mixture by flash chromatography on silica get (30-100% ethyl acetate/hexanes, followed by 10-25% methanol/ethyl acetate) afforded 1-(2-hydroxy-3-propyl-4-{4-[5-(2H-tetrazol-5-yl)-pyridin-2-yloxy]-butoxy}-phenyl)-ethanone as a light yellow oil. ¹H NMR (DMSO-d₆, 500 MHz) δ 12.83 (s, 1H), 8.79 (d, 1H), 8.25 (dd, 1H), 7.80 (d, 1H), 6.98 (d, 1H), 6.66 (d, 1H), 4.41 (m, 2H), 4.17 (m, 2H), 2.57 (s, 3H), 2.54 (m, 2H), 1.99-1.92 (m, 4H), 1.47-1.41 (m, 2H), 0.86 (t, 3H). ESI: 412 (M+H)⁺

### EXAMPLE 50

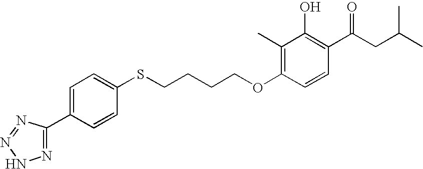

### 1-(2-Hydroxy-3-methyl-4-{4-[4-(2H-tetrazol-5-yl)-phenylsulfanyl]-butoxy}-phenyl)-3-methyl-butan-1-one

Prepared in a similar fashion as outlined in example 6 using 4-cyanothiophenol. ¹H NMR (DMSO-d₆, 500 MHz) δ 12.99 (s, 1H), 7.91 (d, 2H), 7.81 (d, 1H), 7.40 (d, 2H), 6.61 (d, 1H), 4.11 (t, 2H), 3.09 (t, 2H), 2.13-2.11 (m, 1H), 1.94 (s, 3H), 1.91-1.86*m, 2H), 1.79-1.76 (m, 2H), 0.91 (d, 6H). MS (ESI) 441 (M⁺+1).

### EXAMPLE 51

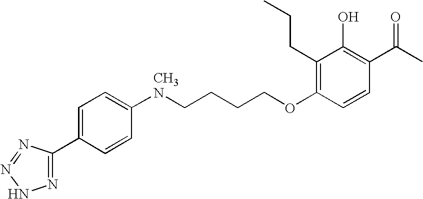

### 1-[2-Hydroxy-4-(4-{methyl-[4-(2H-tetrazol-5-yl)-phenyl]-amino}-butoxy)-3-propyl-phenyl]-ethanone

A mixture of 4-[4-(4-acetyl-3-hydroxy-2-propylphenoxy)butyl]-amino)benzonitrile (80 mg, 0.22 mmol), methanol (4.0 ml) and paraformaldehyde (50 mg, 1.66 mmol) was refluxed overnight and then cooled to room temperature. 10% Palladium on carbon (15 mg) was added to mixture and the mixture was then placed under hydrogen atmosphere at 1 atmosphere pressure. Let mixture stir at room temperature for two hours and the mixture was filtered through celite, washing with ethyl acetate. The collected filtrate was concentrated to give a yellow oil which was further purified by flash chromatography on silica gel (0-50% ethyl acetate/hexanes) to give 4-{[4-(4-acetyl-3-hydroxy-2-propylphenoxy)butyl](methyl)amino}-benzonitrile (15 mg, 18%). A mixture of 4-{[4-(4-acetyl-3-hydroxy-2-propylphenoxy)butyl](methyl)amino}benzonitrile (15 mg, 0.04 mmol), toluene (0.6 ml), dibutyltin oxide (2 mg, 0.01 mmol) and azidotrimethylsilane (0.1 ml, 0.8 mmol) was stirred at 110° C. overnight under nitrogen atmosphere. The reaction mixture was purified by flash chromatography on silica gel (50-100% ethyl acetate/hexanes, followed by 0-10% methanol/ethyl acetate) to afford 1-[2-hydroxy-4-(4-{methyl-[4-(2H-tetrazol-5-yl)-phenyl]-amino}-butoxy)-3-propyl-phenyl]-ethanone as a yellow solid. ¹H NMR (DMSO-d₆, 500 MHz) δ 12.81 (s, 1H), 7.81-7.77 (m, 3H), 6.63 (d, 2H), 6.63 (d, 2H), 4.11 (m, 2H), 3.47 (m, 2H), 2.97 (s, 3H), 2.54 (s, 3H), 2.52 (m, 2H), 1.71-1.60 (m, 4H), 1.43-1.39 (m, 2H), 0.82 (t, 3H). ESI: 424 (M+H)⁺

### EXAMPLE 52

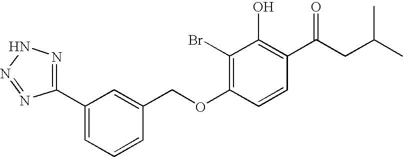

### 1-{3-Bromo-2-hydroxy-4-[3-(2H-tetrazol-5-yl)-benzyloxy]-phenyl}-3-methyl-butan-1-one

Prepared in a similar fashion as outlined in example 1 using 3-(4-bromobutoxy)benzonitrile and 1-(3-Bromo-2,4-dihydroxy-phenyl)-3-methyl-butan-1-one. ¹H NMR (CD₃OD, 500 MHz) δ 13.46 (s, 1H), 8.19 (s, 1H), 8.05 (d, 1H), 8.00 (d, 1H), 7.72-7.63 (m, 2H), 6.89 (d, 1H), 5.48 (s, 2H), 2.94-2.89 (m, 2H), 2.18-2.12 (m 1H), 0.94 (d, 6H). MS (M+H): 430.8.

### EXAMPLE 53

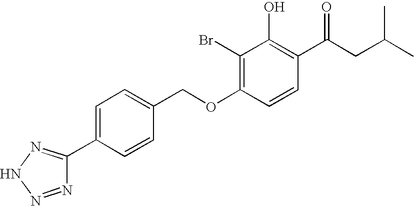

### 1-{3-Bromo-2-hydroxy-4-[4-(2H-tetrazol-5-yl)-benzyloxy]-phenyl}-3-methyl-butan-1-one

Prepared in a similar fashion as outlined in example I using 1-(3-Bromo-2,4-dihydroxy-phenyl)-3-methyl-butan-1-one. ¹H NMR (CD₃OD, 500 MHz) δ 13.46 (s, 1H), 8.10-8.04 (m, 3H), 7.70 (d, 2H), 6.87 (d, 1H), 5.46 (s, 2H), 2.92 (d, 211), 2.19-2.11 (m 1H), 0.95 (d, 6H). MS (M+H): 430.7.

### EXAMPLE 54

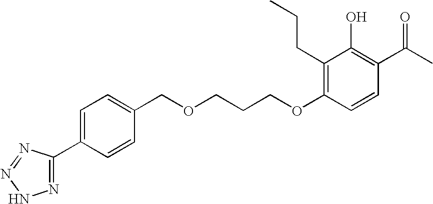

### 1-(2-Hydroxy-3-propyl-4-{3-[4-(2H-tetrazol-5-yl)-benzyloxy]-propoxy}-phenyl)-ethanone

Prepared in a similar fashion as outlined in example 19 using 4-(3-Bromo-propoxymethyl)-benzonitrile. ¹H NMR (DMSO-d₆, 500 MHz), 7.98 (d, 2H), 7.77 (d, 1H), 7.50 (d, 2H), 6.64 (d, 1H), 4.60 (s, 2H), 4.16 (t, 2H), 3.63 (t, 2H), 2.55 (s, 3H), 2.50 (t, 2H), 2.02 (m, 2H), 1.40 (m, 2H), 0.8 (t, 3H). MS (ESI) 410.93 (M⁺+1).

While the invention has been described and illustrated with reference to certain particular embodiments thereof, those skilled in the art will appreciate that various adaptations, changes, modifications, substitutions, deletions, or additions of procedures and protocols may be made without departing from the spirit and scope of the invention. For example, effective dosages other than the particular dosages as set forth herein above may be applicable as a consequence of variations in responsiveness of the mammal being treated for any of the indications with the compounds of the invention indicated above.

1. A compound of the formula I: 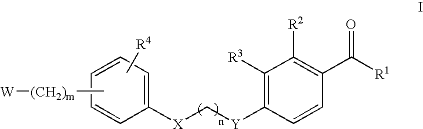
wherein:
W is selected from the group consisting of:
(1) tetrazolyl,
(2) CO₂H,
(3) NHSO₂C₁₋₆alkyl, and
(4) CONHCO—C₁₋₆alkyl;
X is selected from the group consisting of:
(1) —O—,
(2) —S—, and
(3) —NH—,
(4) —N(C₁₋₆alkyl)—,
(5) a bond;
Y is selected from the group consisting of:
(1) —O—, and
(2) —S—;
R¹ is selected from the group consisting of:
(1) C₁₋₆alkyl, which is unsubstituted or substituted with a substituent selected from:
(a) halogen,
(b) hydroxyl, and
(c) phenyl, wherein the phenyl is unsubstituted or substituted with 1-5 substituents independently selected from halogen, cyano, CF3, hydroxyl, C₁₋₆alkyl, and OC₁₋₆alkyl,
(2) C₃₋₇cycloalkyl, which is unsubstituted or substituted with halogen, hydroxyl or phenyl, and
(3) phenyl, wherein the phenyl is unsubstituted or substituted with 1-5 substituents independently selected from halogen, hydroxyl, cyano, CF3, C₁₋₆alkyl, and OC₁₋₆alkyl, wherein the C₁₋₆alkyl and OC₁₋₆alkyl are linear or branched and optionally substituted with 1-5 halogen;
R² is selected from the group consisting of:
(1) hydroxyl,
(2) halogen,
(3) OC₁₋₆alkyl, and
(4) C₁₋₆alkyl, which is unsubstituted or substituted with halogen, hydroxyl or phenyl;
R³ is selected from the group consisting of:
(1) C₁₋₆alkyl, which is unsubstituted or substituted with halogen, hydroxyl or phenyl, and
(2) halogen, and
(3) hydrogen;
R⁴ is selected from the group consisting of:
(1) hydrogen,
(2) halogen, and
(3) C₁₋₆alkyl;
m is an integer selected from 0, 1, 2 and 3;
n is an integer selected from 0, 1, 2, 3, 4, 5 and 6;
and pharmaceutically acceptable salts thereof and individual diastereomers thereof.

2. The compound of claim 1 wherein W is selected from tetrazolyl and CO₂H.

3. The compound of claim 1 wherein X is —O—.

4. The compound of claim 1 wherein Y is —O—.

5. The compound of claim 1 wherein X is a bond and Y is —O—.

6. The compound of claim 1 wherein R¹ is selected from
(1) C₁₋₆alkyl, and
(2) C₅₋₆cycloalkyl.

7. The compound of claim 6 wherein R¹ is selected from
(1) CH₃,
(2) CH(CH₃)₂,
(3) CH₂CH₃,
(4) CH₂CH₂CH₃,
(5) cyclopentyl,
(6) CH₂-cyclopentyl,
(7) phenyl, and
(8) CH_{2phenyl}.

8. The compound of claim 1 wherein R² is selected from hydroxyl and chloro.

9. The compound of claim 1 wherein R³ is selected from
(1) C₁₋₆alkyl,
(2) CH₃,
(3) CH₂CH₃,
(4) CH₂CH₂CH₃, and
(5) chloro.

10. The compound of claim 1 wherein R⁴ is hydrogen.

11. The compound of claim 1 wherein m is 0 or 1.

12. The compound of claim 1 wherein n is selected from 1, 2, 3 or 4.

13. A compound which is selected from the group consisting of: 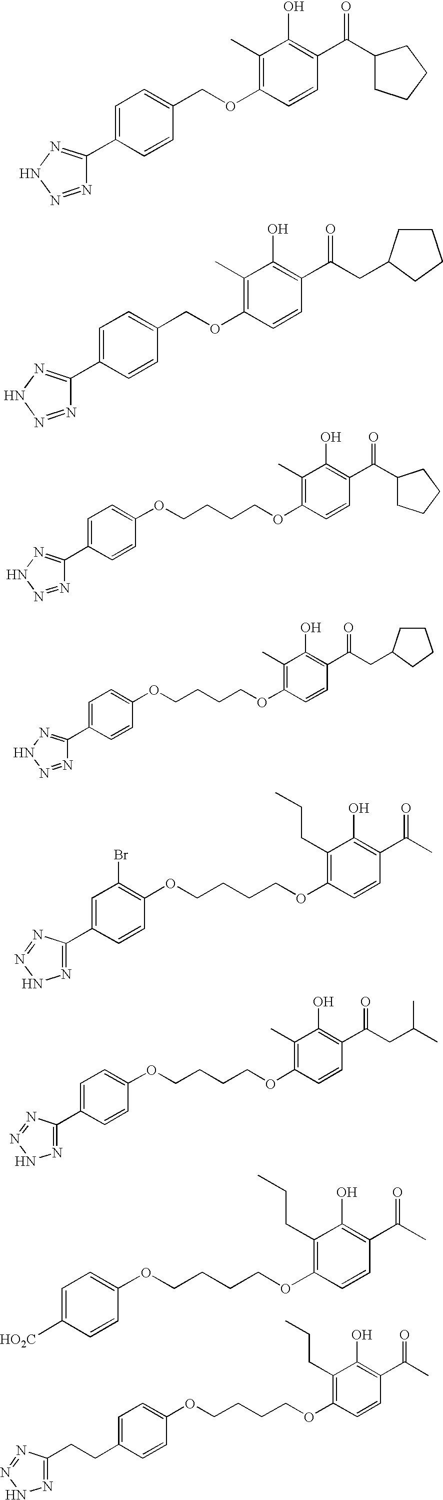 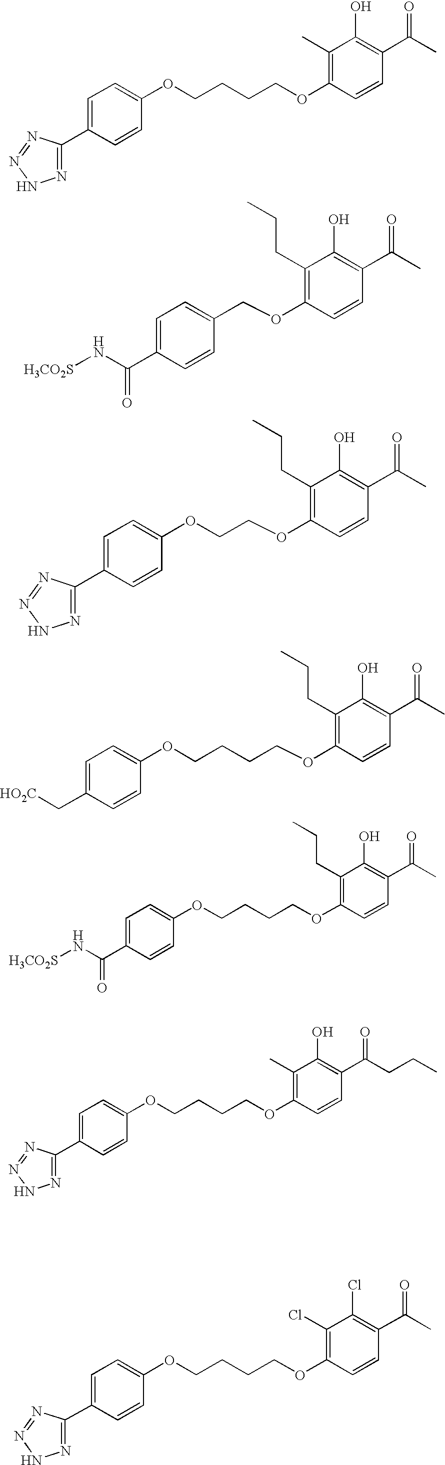 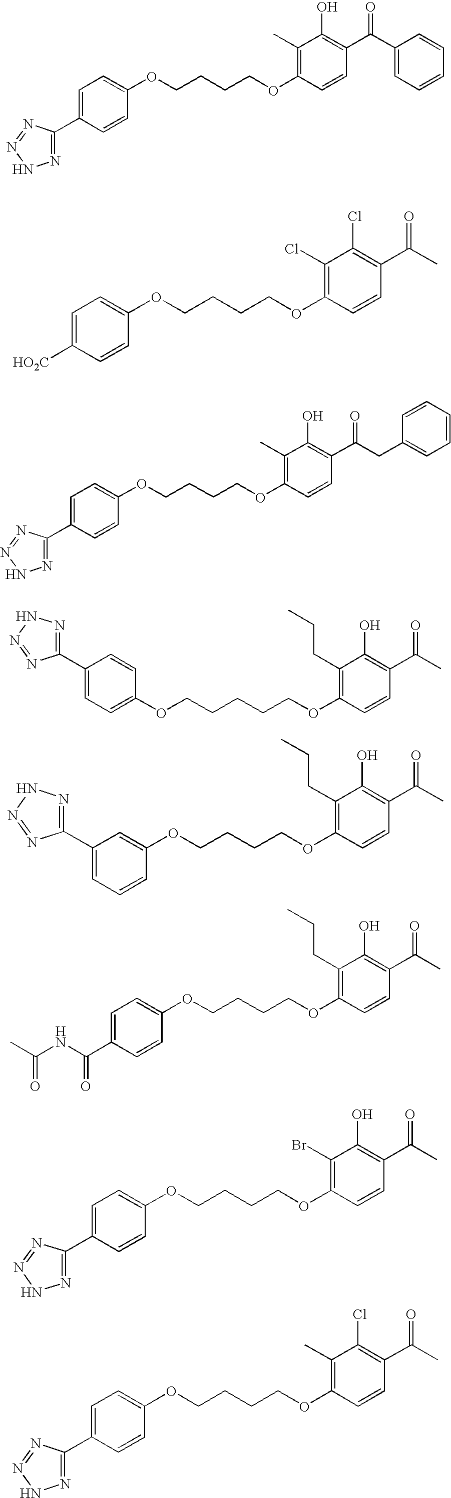 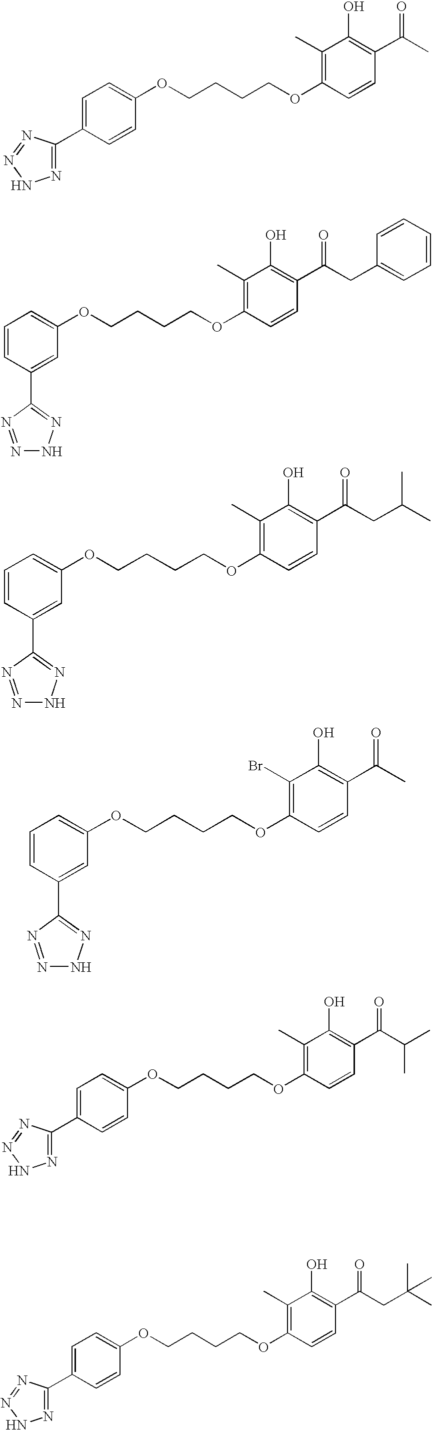 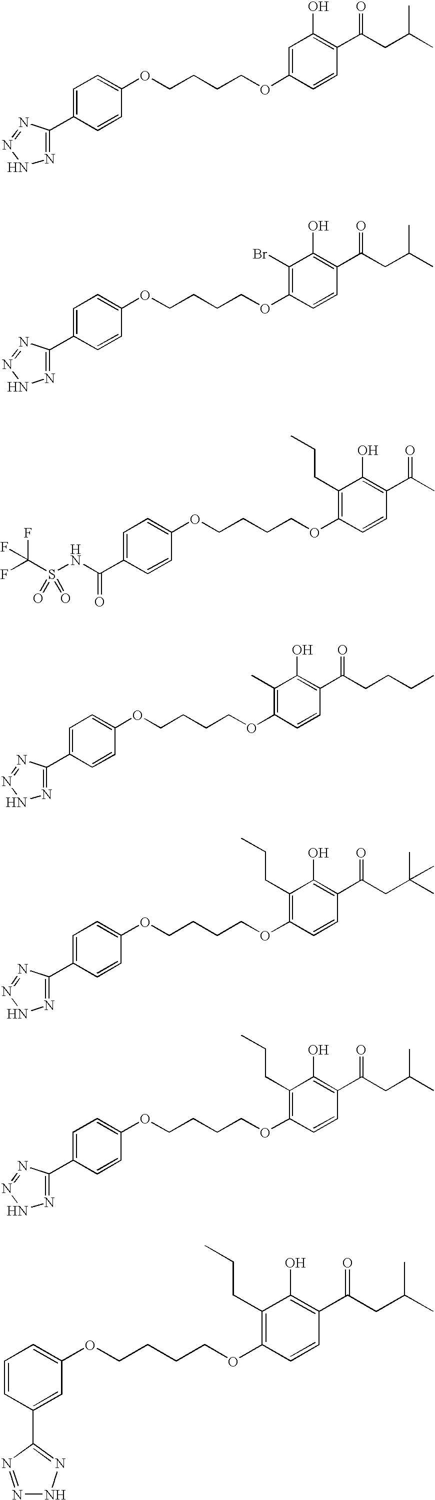 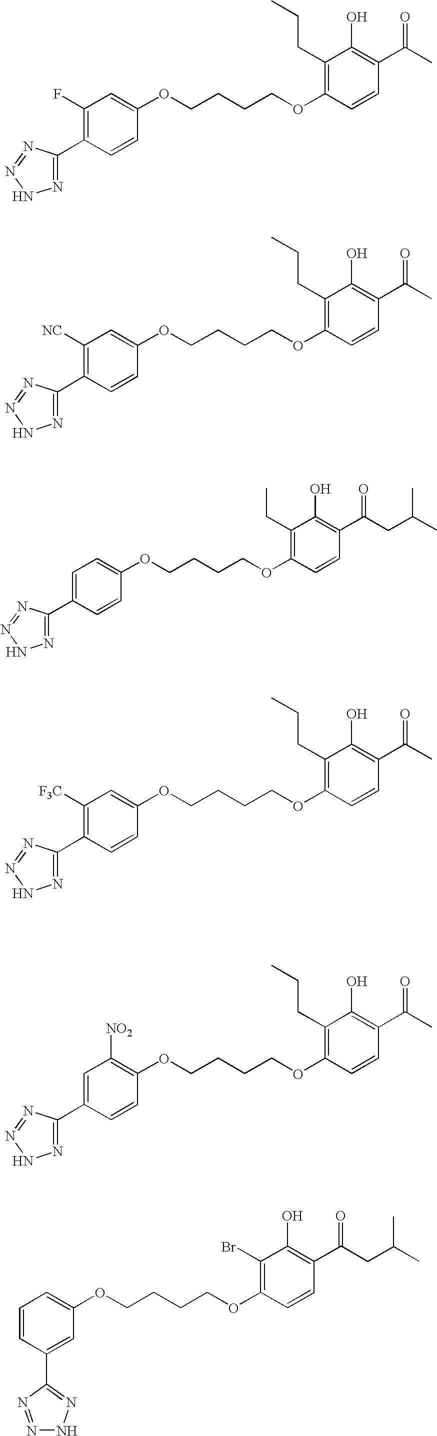 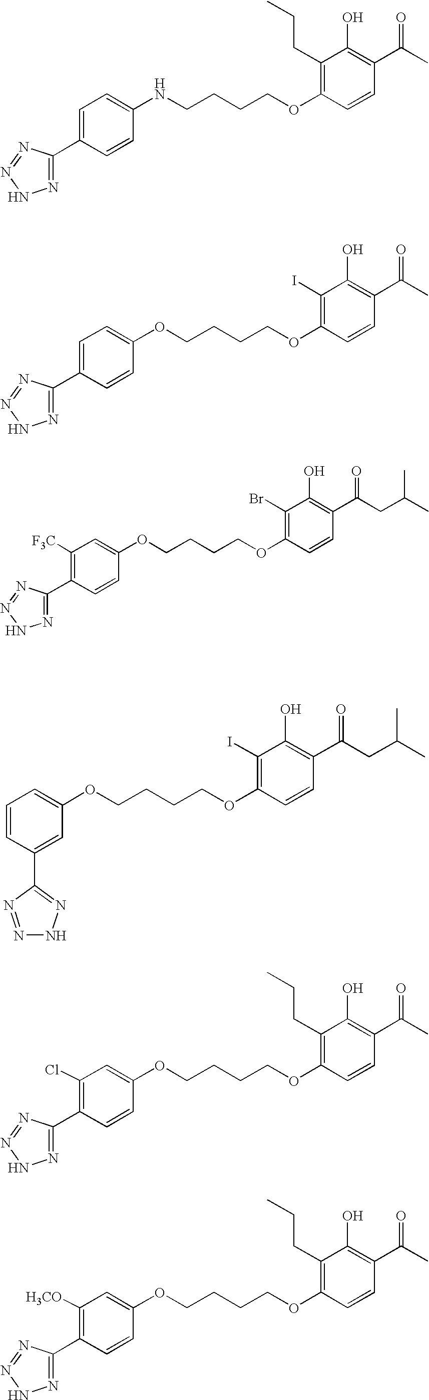 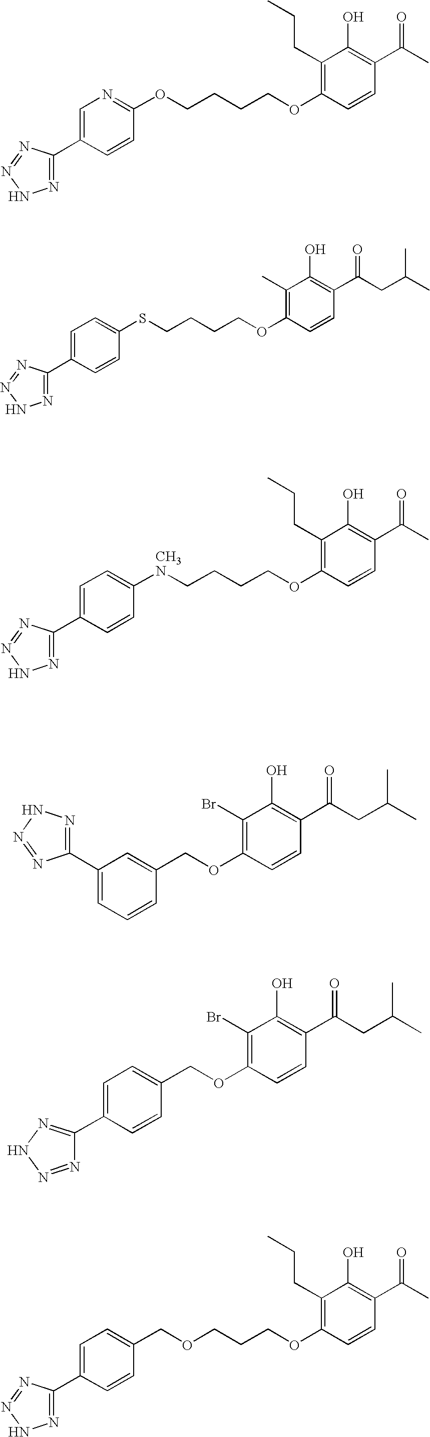
and pharmaceutically acceptable salts thereof.

14. A pharmaceutical composition which comprises an inert carrier and a compound of claim 1.

15. A method for potentiation of metabotorpic glutamate receptor activity in a mammal which comprises the administration of an effective amount of the compound of claim 1.

16. A method for the manufacture of a medicament for potentiation of metabotorpic glutamate receptor activity in a mammal comprising combining the compound of claim 1 with a pharmaceutical carrier or diluent.

17. A method for treating a neurological and psychiatric disorders associated with glutamate dysfunction in a mammalian patient in need of such which comprises administering to the patient a therapeutically effective amount of a compound of claim 1.

18. A method for treating anxiety in a mammalian patient in need of such which comprises administering to the patient a therapeutically effective amount of a compound of claim 1.

19. A method for treating depression in a mammalian patient in need of such which comprises administering to the patient a therapeutically effective amount of a compound of claim 1.

20. A method for treating migraine in a mammalian patient in need of such which comprises administering to the patient a therapeutically effective amount of a compound of claim 1.

21. A method for treating schizophrenia in a mammalian patient in need of such which comprises administering to the patient a therapeutically effective amount of a compound of claim 1.

22. A method for treating epilepsy in a mammalian patient in need of such which comprises administering to the patient a therapeutically effective amount of a compound of claim 1.
